(19) United States
(12) Patent Application Publication
(10) Pub. No.: US 2004/0180046 A1
(43) Pub. Date: Sep. 16, 2004

(54) Bispecific molecules and uses thereof

(76) Inventor: Jeff Himawan, Tampa, FL (US)

Correspondence Address: JONES DAY, 222 EAST 41ST ST, NEW YORK, NY, 10017, US

(21) Appl. No.: 10/258,650

(22) PCT Filed: Apr. 24, 2001
(86) PCT No.: PCT/US01/13161

Related U.S. Application Data
(60) Provisional application No. 60/199,903, filed on Apr. 26, 2000.
(60) Provisional application No. 60/244,812, filed on Nov. 1, 2000.

(51) Int. Cl. A61K 39/395; C07K 16/44

(52) U.S. Cl. 424/132.1; 530/387.3

(57) ABSTRACT

The present invention relates to bispecific molecules that are characterized by having a first binding domain which binds an antigen present in the circulation of a mammal and a second binding domain which binds the C3b-like receptor (known as complement receptor 1 (CR1) or CD35 in primates). The bispecific molecules do not consist of a first monoclonal antibody to CR1 that has been chemically cross-linked to a second monoclonal antibody. The invention also relates to methods of making the bispecific molecules and therapeutic uses thereof, as well as to kits containing the bispecific molecules. The invention further provides polyclonal populations of bispecific molecules, which comprise populations of bispecific molecules with different antigen recognition specificities. Such polyclonal populations of bispecific molecules can be used for targeting multiple epitopes of a pathogenic antigenic molecule and/or multiple variants of a pathogenic antigenic molecule.

### 1. FIELD OF THE INVENTION

The present invention relates to bispecific molecules that are characterized by having a first binding domain which binds an antigen present in the circulation of a mammal and a second binding domain which binds a C3b-like receptor (known as complement receptor 1 (CR1) or CD35 in primates). The invention also relates to methods of making the bispecific molecules and therapeutic uses thereof, as well as to kits containing the bispecific molecules. The invention further relates to polyclonal populations of bispecific molecules.

### 2. BACKGROUND OF THE INVENTION

Antibodies have two principal functions, the first is to opsonize an antigen, i.e., recognize and bind the antigen, and the second is to mobilize other elements of the immune system to destroy the antigen. Pathogenic antigenic molecules in the circulatory system are thought to be cleared by fixed tissue macrophages in the liver and spleen, i.e., the reticuloendothial system (RES). Antibodies enhance the delivery and recognition of antigens to the RES; however, enhanced delivery of target antigens to phagocytes for clearance by a specific antibody (i.e., a specific immunoglobulin) to said antigen is not always sufficient for rapid and efficient clearance of the antigen.

Circulating pathogenic antigenic molecules cleared by the fixed tissue phagocytes may include any antigenic moiety. Failure of the immune system to effectively remove the pathogens and/or toxins from the mammalian circulation can lead to traumatic and hypovolemic shock (Altura and Hershey, 1968, Am. J. Physiol. 215:1414-9).

The clearance of antigens from the circulation involves the binding of the antigen to a receptor on a phagocyte and the subsequent removal of the antigen from the circulation. Antigens are endocytosed by phagocytes and the antigens are subsequently destroyed by chemical and/or proteolytic degradation in the phagocyte.

The antigen's rate and efficiency of removal from the circulation is dependent upon multiple factors including the number of fixed tissue phagocytes present in the organism, the number of appropriate receptors on the fixed tissue phagocytes, the serum concentration of opsonins, the affinity of the receptor for the pathogen, and the concentration of the pathogens (Reichard and Filkins, 1984, The Reticuloendothelial System; A Comprehensive Treatise, pp. 73-101 (Plenum Press)).

Serum opsonins, such as antibodies or complement, enhance the clearance of a pathogen by binding to the pathogen and coating it so that it is more readily bound by receptors on phagocytes. For example in primates, the complement factor C3b clears pathogens by binding to an immune complex. The C3b/immune complex then binds to a C3b receptor, which is expressed on the surface of a hematopoietic cell, e.g., on erythrocytes in primates, via the C3b molecule attached to the immune complex. The complex is then chaperoned by the hematopoietic cell to the RES for clearance. To demonstrate this clearance mechanism, Johnson et al. pre-coated agarose beads with C3b and showed that the coated beads were cleared more rapidly from the circulation than uncoated beads (1983, Scand. J. Immunol., 17:403).

Any moiety that can bind an antigen and is itself bound by immune cells can serve as an opsonin. A significant limitation on the rate of clearance of pathogens from the circulation is low concentration of opsonins in the serum. The low number of opsonins relative to the number of pathogens present in the bloodstream allows many of the pathogens to escape prompt and efficient clearance (Reichard and Filkins, 1984, The Reticuloendothelial System; A Comprehensive Treatise, pp. 73-101 (Plenum Press)).

Numerous techniques have been developed which identify potential binding moieties, i.e., opsonins, to pathogens in the hopes that these binding moieties will have utility as a therapeutic agent against the pathogen. For example combinatorial chemistry, or phage display libraries have been used extensively to identify binding moieties for potential therapeutic uses.

A significant weakness of the phage display and combinatorial chemistry techniques is that although the identified binding domain may interact with the pathogen, the binding domain may not have a therapeutic utility. For example, binding moieties derived from the foregoing techniques rarely direct the immune system to attack the pathogen and clear it from the circulation as would naturally occurring opsonins such as antibodies or complement. Another limitation of the identified binding domain is that there is no reasonable expectation that it will interfere with the normal replication of the pathogen in the circulation, thereby therapeutically treating the subject by blocking the growth or perpetuation of the pathogen.

The development of monoclonal antibody technology, first disclosed by Kohler and Milstein (1975, Nature 256:495-497), has allowed the generation of a nearly unlimited supply of antibodies of precise and reproducible specificity. The Kohler and Milstein procedure involves the fusion of spleen cells obtained from an immunized animal with an immortal myeloma cell line which results in a population of hybridoma cells, which will include a hybridoma that produces an antibody of the desired specificity. The hybridoma which produces an antibody having the requisite specificity is then selected, or ‘cloned’, from this population of hybridomas using conventional techniques such as enzyme linked immunosorbent assays (ELISA).

Additional approaches to generating antibodies useful for therapeutics have been developed as an alternative to the laborious immunization procedure mentioned above. One approach entails cloning a sub-library of genes that encode an antibody in frame with phage structural proteins, then inserting these recombinant genes into bacteriophage, which will express the antibody-structural fusion protein on the virus surfaces as described in Clackson et al., 1991, Nature 352:624; Marks et al., 1992, J. Mol. Biol. 222:581; Zebedee et al., 1992, Proc. Natl. Acad. Sci. USA 39:3175; Gram et al., 1992, Proc. Natl. Acad. Sci. USA 89:3576. However, the production of an antibody that binds a pathogen of interest does not always result in a therapeutically effective antibody.

Because antibodies are generally inadequate therapeutic agents by themselves, monoclonal antibody technology has been further modified to generate antibodies where the two variable regions have distinct antigen binding properties. The bispecific antibodies are potentially more useful than monoclonal antibodies, for example, they can target two separate antigens and bring a therapeutic agent into proximity to a target pathogen; however, these bispecific antibodies also contain the same inherent limitations as the parental antibodies in that they have no special therapeutic properties (for review, see Songsivilai and Lachmann, 1990, Clin. Exp. Immunol., 79:315-321; and Songsivilai and Lachmann, 1995, Monoclonal Antibodies, Cambridge University Press, pp. 121-141).

A need exists for a method of treating a subject with a therapeutic molecule, such that upon the therapeutic molecule contacting a pathogenic antigenic molecule, the pathogenic antigenic molecule is efficiently cleared from the circulation. To this end, Taylor et al. have shown that extracellular chemical crosslinking of a first monoclonal antibody specific to a pathogenic antigen to a second monoclonal antibody specific to a primate C3b receptor creates a bispecific heteropolymeric antibody which can rapidly and efficiently bind and clear a pathogenic antigenic molecule from a primate's circulation (U.S. Pat. Nos. 5,487,890 and 5,470,570; FIG. 1, panel B).

The present invention provides compositions and methods for treatment or prevention of diseases using bispecific molecules that bind both a C3b-like receptor, or its functional equivalent, and an antigen to be cleared from the circulation. The binding of a C3b-like receptor by a bispecific immunadhesin of the present invention tethers the antigen to a hematopoietic cell which then chaperones the antigen to its destruction by the reticuloendothelial system.

### 3. SUMMARY OF THE INVENTION

The present invention relates to bispecific molecules that are characterized by having a first binding domain which binds-an antigen present in the circulation of a mammal and a second binding domain which binds a C3b-like receptor or its functional equivalent (known as complement receptor 1 (CR1) or CD35 in primates). The invention also relates to methods of making the bispecific molecules and therapeutic and prophylactic uses thereof, as well as to kits containing the bispecific molecules, and nucleic acids encoding the bispecific molecules that are polypeptides, cells transformed with the nucleic acids, and recombinant methods of production of the bispecific molecules.

The present invention represents a significant improvement over the limitations of earlier described techniques. In particular, the present inventor has determined that bispecific antibodies, specific to both a C3b-like receptor and an antigen to be cleared from the circulation, could be rapidly and efficiently cleared from the mammalian circulation. Bispecific molecules can include any single polypeptide or any multi-subunit polypeptide which has a first binding domain specific for a C3b-like receptor and a second binding domain specific for an antigen of interest. The bispecific molecules of the invention do not consist of a first monoclonal antibody to CR1 that has been chemically cross-linked to a second monoclonal antibody. Thus, the multi-subunit polypeptide is preferably not chemically crosslinked to form the bispecific molecule, therefore, reducing the antigenicity of the molecule.

As used herein, the term C3b-like receptor is understood to mean any mammalian circulatory molecule which has an analogous function to a primate C3b receptor, for example CR1.

In a preferred embodiment, the bispecific molecule is a bispecific immunoglobulin wherein the first variable region binds an antigenic molecule to be cleared from the circulation and the second variable region binds a C3b-like receptor. More preferably, the C3b-like receptor is the C3b receptor of a primate (see, FIG. 1, panel C). In a specific embodiment, such an immunoglobulin is chimeric by virtue of having a human constant region, and/or is humanized.

The humanized bispecific antibodies should be poorly recognized as foreign proteins by the human immune system, that is, they are poorly immunogenic, thus making them preferable for therapeutic or diagnostic use in humans. In particular, a human immune reaction would diminish the therapeutic effectiveness of the bispecific antibodies with regard to repeated treatments. Additionally, the bispecific antibodies are preferably not produced by the use of extracellular crosslinking agents which can both denature antibodies reducing the yield of bispecific molecule, and also may act as an immunogenic hapten and thereby reduce the utility of repeated administration of the humanized bispecific antibody.

In a specific embodiment, a nucleic acid is provided that comprises sequence(s) encoding a bispecific molecule of the invention, operatively linked to a promoter (e.g., a heterologous promoter). The nucleic acid can be intrachromosomal, or a vector (e.g., a plasmid vector, particularly a plasmid expression vector). Methods of recombinant production are also provided, comprising culturing a host cell transformed with such a nucleic acid such that the encoded bispecific molecule is expressed, and, when the bispecific molecule is a polypeptide multimer composed of separate polypeptides, assembles together within the cell, and recovering the expressed bispecific molecule. When the bispecific molecule is a polypeptide multimer (e.g., an immunoglobulin), alternatively, its monomeric components can be expressed in the same host cell or different host cells, purified, and then combined in vitro to form the bispecific molecule.

In one embodiment, the bispecific molecule is a single polypeptide which has a first binding domain (BD1), such as an antibody variable domain or a receptor ligand, fused to the amino terminus of a Fc domain, namely a hinge region, a CH2 domain and a CH3 domain, of an immunoglobulin heavy chain which in turn is fused to a second binding domain (BD2) at its carboxy terminus. Alternatively, the bispecific molecule is composed of two separate, associated fusion polypeptides, the first having a BD1 at the amino terminus of a CH2 and CH3 portion of an immunoglobulin heavy chain, and the second polypeptide comprising a CH2 and CH3 portion of an immunoglobulin heavy chain with a BD2 fused to its carboxy terminus. Alternately, the binding domains can be switched from the carboxy or amino terminus of the respective Fc domain. These two polypeptides form a dimer via interaction of the heavy chain domains when expressed in the same cell, or alternatively, each polypeptide can be expressed in separate cells followed by in vitro joining, as discussed below.

In another embodiment, the bispecific molecule of the invention consists of two associated polypeptides wherein the binding domains are single chain Fv domains (scFv's). A scFv comprises a variable light chain fused to a variable heavy chain via a connecting peptide. The first polypeptide consists essentially of a scFv with specificity for a C3b-like receptor fused to the amino terminus of an immunoglobulin Fc domain. The second polypeptide consists essentially of a scFv with specificity for an pathogenic antigenic molecule, fused to the carboxy terminus of an immunoglobulin Fc domain. The invention also contemplates that the scFv domains can be at either the carboxy or amino terminal ends of the Fc domains. These two polypeptides form a dimer via interaction of the heavy chain domains when expressed in the same cell, or they are expressed in separate cells followed by in vitro assembly together, as discussed below.

In another embodiment, the bispecific molecule is a single recombinant polypeptide containing a first variable heavy chain, a first variable light chain, CH2, CH3, a second variable heavy chain, and a second variable light chain. The first variable heavy and light chains are specific for a C3b-like receptor and the second variable heavy and light chains are specific for a pathogenic antigenic molecule.

In a preferred embodiment, the invention provides a method of treating a mammal having an undesirable condition associated with the presence of a pathogenic antigenic molecule comprising administering to the mammal a therapeutically effective dose of a bispecific molecule, which bispecific molecule (a) does not consist of a first monoclonal antibody to CR1 that has been chemically crosslinked to a second monoclonal antibody, (b) comprises a first binding domain which binds said pathogenic antigenic molecule, and (c) comprises a second binding domain which binds a C3b-like receptor of the mammal.

In various embodiments, the invention provides kits comprising in one or more containers a bispecific molecule, nucleic acid(s) encoding a bispecific molecule, and cells transformed with such nucleic acid(s). In a specific embodiment, the invention provides a kit comprising in one or more containers a first vector and a second vector, said first vector comprising a first DNA sequence encoding at least a first immunoglobulin variable heavy chain domain fused via a polypeptide linker to a first immunoglobulin variable light chain domain, and said second vector comprising a second DNA sequence encoding at least a second immunoglobulin variable heavy chain domain fused via a olypeptide linker to a second immunoglobulin variable light chain domain, wherein said first immunoglobulin variable heavy chain domain and said first immunoglobulin variable light chain bind a pathogenic antigenic molecule, and said second immunoglobulin variable heavy chain domain and second immunoglobulin variable light chain domain bind a C3b-like receptor.

In another embodiment, the invention provides a cell transformed with one or more recombinant vectors encoding a bispecific molecule. In a more particular embodiment, the cell contains one recombinant nucleic acid expressing a polypeptide with binding specificity for both a C3b-like receptor and a pathogenic molecule and is capable of being cleared by the reticuloendothelial system. In another specific embodiment, the transformed cell contains more than one nucleic acid, wherein one of the nucleic acids encodes a first binding domain with specificity to a C3b-like receptor, and a second nucleic acid encodes a second binding domain with specificity for a pathogenic antigenic molecule, the two polypeptides being capable of associating together through, for example a hinge region which mediates associating of heavy chains of an antibody, and also being capable of binding the C3b-like receptor and pathogenic antigenic molecule through their respective binding domains.

In another embodiment, the invention provides a method of producing a bispecific immunoglobulin-secreting cell which has a first antigen recognition region which binds to a C3b-like receptor and a second antigen recognition region which binds to a pathogenic antigenic molecule, comprising the steps of fusing a first cell expressing an immunoglobulin which binds to the C3b-like receptor with a second cell expressing an immunoglobulin which binds to the pathogenic antigenic molecule, and selecting for cells that express the bispecific immunoglobulin.

In another embodiment, the invention provides a transformed cell containing at least two vectors, at least one of said vectors comprising a first DNA sequence encoding at least a first variable heavy chain and light chain and at least another one of said vectors comprising a second DNA sequence encoding at least a second variable heavy and light domain, said first heavy chain and first light chain capable of binding a pathogenic molecule, and said second heavy chain and second light chain capable of binding a C3b-like receptor expressed on a cell.

In another embodiment, the invention provides a method of preventing an undesirable condition (e.g., disease, disorder) associated with the presence of a pathogenic antigenic molecule in a mammal, comprising administering prior to the onset of the undesirable condition, to the mammal a prophylactically effective amount of a bispecific molecule, which bispecific molecule (a) does not consist of a first monoclonal antibody to CR1 that has been chemically cross-linked to a second monoclonal antibody, (b) comprises a first binding domain which binds said pathogenic antigenic molecule, and (c) comprises a second binding domain which binds a C3b-like receptor of the mammal.

In another embodiment, the invention provides a method of treating a mammal having an undesirable condition associated with the presence of a pathogenic antigenic molecule, and which is not composed of two monoclonal antibodies or fragments thereof chemically crosslinked to each other, comprising the steps of contacting a bispecific molecule which has a first antigen recognition domain which binds a C3b-like receptor and has a second antigen recognition domain which binds a pathogenic antigenic molecule with hematopoietic cells from a mammal, to form a hematopoietic cell/bispecific molecule complex, and administering the hematopoietic cell/bispecific molecule complex to the subject in a therapeutically effective amount.

In another embodiment, the invention provides a method for treating a mammal having an undesirable condition associated with the presence of a pathogenic antigenic molecule, and which is not composed of two monoclonal antibodies or fragments thereof chemically crosslinked to each other, comprising the steps of administering a hematopoietic cell/bispecific molecule complex to the subject in a therapeutically effective amount, said complex consisting essentially of a hematopoietic cell bound to one or more bispecific molecules, said bispecific molecule having a first antigen recognition domain which binds a C3b-like receptor on the hematopoietic cell and a second antigen recognition domain which binds a pathogenic antigenic molecule, said bispecific molecule not being composed of two monoclonal antibodies or fragments thereof chemically crosslinked to each other.

In another embodiment, the invention provides a method for producing a bispecific molecule comprising at least a first antigen recognition region which binds a C3b-like receptor and a second antigen recognition region which binds a pathogenic antigenic molecule or fragment thereof comprising the steps of transforming a cell with a first DNA sequence encoding at least the first antigen recognition region and a second DNA sequence encoding at least the second antigen recognition region, and independently expressing said first DNA sequence and said second DNA sequence so that said first and second antigen recognition regions are produced as separate molecules which assemble together in said transformed single cell, whereby a bispecific molecule that is not two separate monoclonal antibodies chemically crosslinked to each other and that is capable of binding to a C3b-like receptor with a first antigen recognition region and also capable of binding an antigen to be cleared from the circulation with a second antigen recognition region is formed.

The present invention also relates to polyclonal populations comprising a plurality of different bispecific molecules and their production and uses. Preferably, the plurality of bispecific molecules in a polyclonal population includes specificities for different epitopes of an antigenic molecule and/or for different variants of an antigenic molecule. More preferably, the plurality of bispecific molecules of the polyclonal population includes specificities for the majority of naturally-occurring variants of an antigenic molecule. Polyclonal populations of bispecific molecules that target multiple variants of a pathogen or multiple pathogens are also envisioned. In preferred embodiments, at least 90%, 75%, 50%, 20%, 10%, 5%, or 1% of bispecific molecules in the polyclonal population target the desired antigenic molecule and/or antigenic molecules. In other preferred embodiments, the proportion of any single bispecific molecule in the polyclonal population does not exceed 90%, 50%, or 10% of the population. The polyclonal population comprises at least 2 different bispecific molecules with different specificities. More preferably, the polyclonal population comprises at least 10 different bispecific molecules with different specificities. Most preferably, the polyclonal population comprises at least 100 different bispecific molecules with different specificities.

In some embodiments of the invention, a population of bispecific molecules is produced by transfecting a hybridoma cell line that expresses an immunoglobulin that binds a C3b-like receptor with a population of eukaryotic expression vectors containing nucleic acids encoding the heavy and light chain variable regions of a polyclonal population of immunoglobulins that have different binding specificities. In a preferred embodiment, a phage display library is first screened to select a polyclonal sublibrary having binding specificities directed to the antigenic molecule or antigenic molecules of interests by affinity chromatography. The nucleic acids encoding the heavy and light chain variable regions are then linked head to head to generate a library of bidirectional phage display vectors. The bidirectional phage display vectors are then transferred in mass to bidirectional mammalian expression vectors which are used to transfect the hybridoma cell line.

In another preferred embodiment, a polyclonal population of bispecific molecules is obtained by affinity screening of a phage display library having a sufficiently large repertoire of specificities with an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then co-expressed with vectors containing a nucleotide sequence encoding an anti-CR1 binding domain in a suitable host. Alternatively, the population of expression vectors and the vectors containing a nucleotide sequence encoding an anti-CR1 binding domain are expressed in separate hosts and the antigen binding domains and the anti-CR1 binding domain are combined in vitro to form the polyclonal population of bispecific molecules.

In other embodiments of the invention, the polyclonal populations of bispecific molecules are produced recombinantly, whereby the polyclonal population of nucleotides which encode antibody variable domains with the desired binding specificities are fused to nucleotides which encode immunoglobulin constant domain sequences and expressed in a suitable host. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and heavy chain.

Polyclonal populations of bispecific molecules comprising single polypeptide bispecific molecules can be produced recombinantly. A polyclonal population of nucleic acids encoding a polyclonal population of selected antigen recognition regions is fused to nucleic acids encoding the antigen recognition region that binds a C3b-like receptor to obtain a population of nucleic acids encoding a population of bispecific molecules. The population of bispecific molecules are then expressed in a suitable host to produce a polyclonal population of bispecific molecules.

It is believed that bispecific antibodies may have the added property of slow clearance from the circulation when not bound to an antigen (see, for example, Craig et al., 1999, Clinical Immunology, 92:170-180); this property is especially useful when the bispecific antibodies are used prophylactically.

### 4. DESCRIPTION OF THE FIGURES

FIGS. 1A-C illustrate production of bispecific antibodies. Panel A depicts two separate monoclonal antibodies produced by separate hybridomas. mAb1 binds the c3b receptor, and mAb2 binds Ag2. Panel B depicts the traditional method of extracellular chemically cross-linking of monoclonal antibodies to generate heteropolymers. The wavy line is a representation of an extracellular chemical crosslinking agent. Panel C depicts a bispecific molecule of the invention, that is a bispecific immunoglobulin created by the fusion of the hybridomas producing the antibodies shown in Panel A; the left arm of the antibody as depicted binds c3b receptor; the right arm binds Ag2.

FIG. 2 graphically depicts the domains of an immunoglobulin molecule, and the cleavage sites in an immunoglobulin upon protease digestion with papain or pepsin.

FIG. 3 illustrates the ten possible combinations of immunoglobulin antibodies formed upon fusion of two different hybridomas which secrete monoclonal antibodies.

FIGS. 4A-F illustrate bispecific molecule embodiments of the invention. Left to right (or top to bottom in FIGS. 4C and 4D) depicts amino- to carboxy-terminal order. Panel A depicts a bispecific molecule which is a single polypeptide consisting essentially of a first binding domain (BD1), fused to the amino terminus of a CH2 and CH3 portion of an immunoglobulin heavy chain fused to a second binding domain (BD2) at its carboxy terminus. Panel B depicts a dimer consisting of a first polypeptide consisting essentially of a BD1 fused to the amino terminus of a Fc domain of an antibody(a hinge region, a CH2 domain and a CH3 domain) and a second polypeptide consisting essentially of a Fc domain with a BD2 domain fused to the Fc domain's carboxy terminus. Panel C depicts the structure, in a specific embodiment, of one or both of the polypeptides of the dimer of Panel B. Panel C depicts a polypeptide that consists essentially of a variable light chain domain (VL) and constant light chain domain (CL) fused via a linker molecule to the amino terminus of a VH domain followed by a CH1 domain, a hinge region, a CH2 domain and a CH3 domain. Panel D depicts the structure, in a specific embodiment, of one or both of the polypeptides of the dimer of Panel B. Panel D depicts a polypeptide containing a scFv fused to the amino terminus of a CH1 domain, followed by a hinge region, a CH2 domain and a CH3 domain. Panel E depicts a polypeptide comprising two separate scFv with specificity for two separate antigens, the polypeptide consisting essentially of a first scFv domain fused to a CH2 domain, followed by a CH3 domain, and a second scFv domain. “a” indicates “binds to.” Panel F depicts a polypeptide comprising two variable regions with specificity for two separate antigens, the polypeptide consisting essentially of a first variable heavy chain fused to a variable light chain, a CH2 domain, a CH3 domain, a variable heavy chain and variable light chain.

### 5. DETAILED DESCRIPTION OF THE INVENTION

The present invention relates to bispecific molecules, more particularly to bispecific antibodies, which are characterized by having a first antigen recognition region which binds an antigenic molecule to be cleared from a subject (a pathogenic antigenic molecule) and a second antigen recognition region which binds a C3b-like receptor or its functional equivalent. The C3b receptor is known as the complement receptor 1 (CR1) in primates or CD35. As used herein, the term C3b-like receptor is understood to mean any mammalian circulatory molecule which has an analogous function to a primate C3b receptor, for example CR1.

The bispecific molecules of the invention do not consist of a first monoclonal antibody to CR1 that has been chemically cross-linked to a second monoclonal antibody.

Extracellular chemical crosslinking of polypeptides has significant disadvantages. First, the chemical crosslinking process can denature polypeptides thus increasing the dose necessary for effective treatment, and second, the crosslinking reagent may act as an immunogenic hapten.

Immune recognition of the crosslinking agent covalently bound to the bispecific molecule could significantly reduce the utility of repeated administration of the bispecific molecule and other therapeutic molecules that use the same cross-linking agent. Thus, preferably, extracellular chemical cross-linking (other than disulfide bond formation), particularly by use of heterofunctional reagents, is avoided in producing the bispecific molecules of the invention.

In a specific embodiment of the invention, neither the first antigen recognition region that binds an antigenic molecule not the second antigen recognition region that binds a C3b-like receptor in a bispecific molecule comprises more than one heavy and light chain pair.

The complement component, C3b, is the ligand for the C3b receptor and is activated to bind cells, or immune complexes (IC), which are targeted for clearance by the immune system. The C3b component, after binding the targeted cell or IC, subsequently binds the C3b receptor, thereby tethering the antigen, e.g., a cell or an IC, to the circulating red blood cell in a complex. This red blood cell-antigen complex then passes through the circulation to the liver or spleen and the complex is then thought to be recognized and eliminated by the reticuloendothelial system. The antigen is then phagocytosed by macrophages in the reticuloendothelial system, and the red blood cell is released back into the circulation (Cornacoff, J., et al., 1983, J. Clin. Invest., 71:236-47).

The bispecific molecules of the present invention utilize the unique properties of the C3b-like receptor, expressed on the surface of hematopoietic cells (for example, CR1 on erythrocytes in humans), to clear circulating antigens. In particular, the compositions of the present invention are useful for rapidly and efficiently clearing antigens from the circulation.

The compositions and methods of the invention are useful for the treatment of diseases, disorders, or other conditions wherein an antigenic molecule is desired to be removed from the circulation (i.e., where the antigenic molecule is, or is a component of, a causative agent of the condition), as well as for the prevention of the onset of the symptoms and signs of such conditions, or for the delay of the symptoms and signs in the evolution of these conditions. The methods of the invention will be, for example, useful for the treatment of such conditions, including the improvement or alleviation of any symptoms and signs of such conditions, the improvement of any pathological or laboratory findings of such conditions, the delay of the evolution of such conditions, the delay of onset of any symptoms and signs of such conditions, as well as the prevention of occurrence of such conditions, and the prevention of the onset of any of the symptoms and signs of such conditions.

The preferred subject for administration of a bispecific antibody of the invention, for therapeutic or prophylactic purposes, is a mammal including but not limited to non-human animals (e.g., horses, cows, pigs, dogs, cats, sheep, goats, mice, rats, etc.), and in a preferred embodiment, is a human or non-human primate.

Preferred characteristics of a mammal treated with the methods and compositions of the present invention include sufficient volume of blood flow to the liver to provide rapid and efficient clearance of the pathogenic antigenic molecule, and also the presence of fixed tissue macrophages in the liver and spleen (e.g., Kupffer cells). Antigen clearance is relatively independent of the animal species, rather, antigen clearance depends on the animal size, total macrophage cell numbers, and the dose of the therapeutic.

Although the examples disclosed herein are carried out using mouse mAbs, as discussed below (Sections 6-6.2), currently available technology allows “humanization” of these mouse mAbs. This will decrease the chance that in a human, an immune response to the bispecific antibody will diminish its effectiveness in repeated doses due to human anti-mouse antibodies (HAMA). More preferably, human antibodies are used to create the bispecific antibodies of the invention (see Section 5.1.1.2).

### 5.1. Bispecific Antibodies

In a preferred embodiment discussed below (Section 5.1.2), bispecific molecules are bispecific antibodies which are produced by fusion of two hybridoma cell lines (Hybrid Hybridoma). Fusion of two hybridomas results in up to ten different antibody products. The ten different antibodies result from association of the different heavy and light chain genes produced. However, the bispecific antibody is readily purified in quantities sufficient for use as an immunotherapeutic using standard column chromatography, cell sorting or immuno-purification schemes as described below (Section 5.2).

In yet another embodiment, bispecific antibodies are produced by introduction of antibody genes by transfection into a system to recombinantly express bispecific antibodies in, for example fibroblasts, hybridomas, myelomas, insect cells, or any protein expression system.

In yet another embodiment, bispecific antibodies are produced by isolation of the individual monoclonal antibodies, breaking of disulfide linkages of each specific antibody and subsequent recombination of antibody heavy and light chain polypeptides in vitro (see, for example Arathoon et al., WO 98/50431).

5.1.1 Antibodies

The term “antibody” as used herein refers to immunoglobulin molecules. The invention also envisions the use of antibody fragments that contain an antigen binding site which specifically binds an antigen, such as an antigen of the invention. Examples of immunologically active fragments of immunoglobulin molecules include F(ab) and F(ab′)2 fragments which can be generated by treating the antibody with an enzyme such as pepsin or papain. Examples of methods of generating and expressing immunologically active fragments of antibodies can be found in U.S. Pat. No. 5,648,237 which is incorporated herein by reference in its entirety.

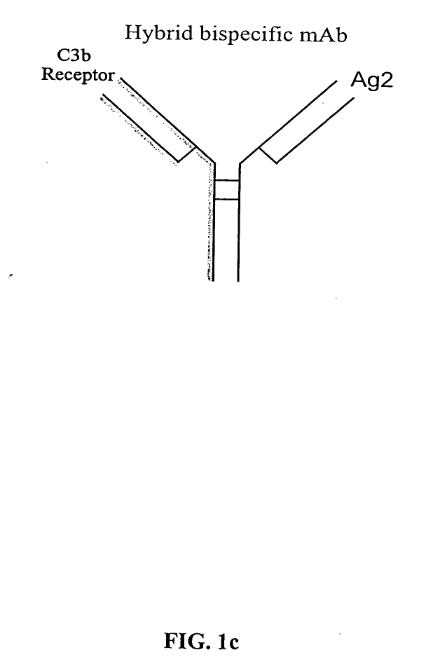

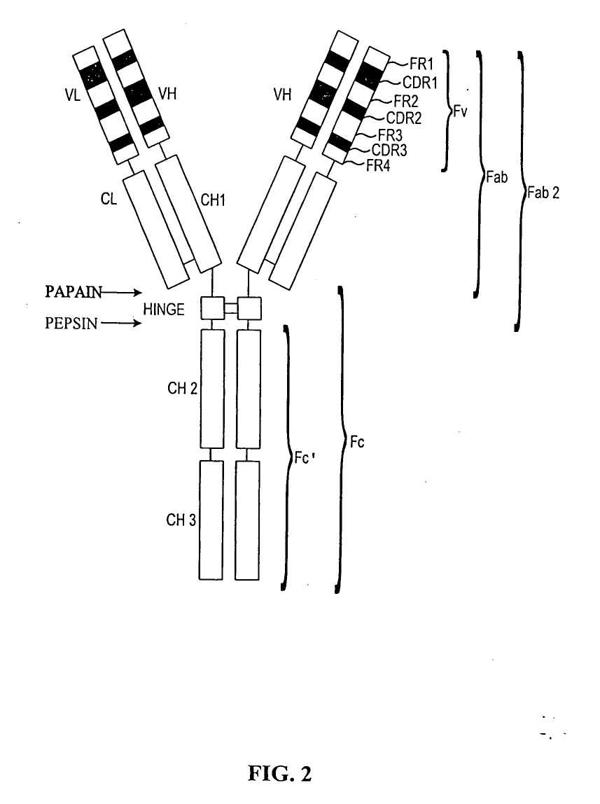

The immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (VL) and a constant light (CL) domain (FIG. 2). Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains (FIG. 2). The IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain (FIG. 2).

A chimeric antibody may be made by splicing the genes from a monoclonal antibody of appropriate antigen specificity together with genes from a second human antibody of appropriate biologic activity. More particularly, the chimeric antibody may be made by splicing the genes encoding the variable regions of an antibody together with the constant region genes from a second antibody molecule. This method is used in generating a humanized monoclonal antibody wherein the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337 which are incorporated herein by reference in their entirety).

A bispecific antibody suitable for use in the present invention may be obtained from natural sources or produced by hybridoma, recombinant or chemical synthetic methods, including modification of constant region functions by genetic engineering techniques (U.S. Pat. No. 5,624,821). The bispecific antibody of the present invention may be of any isotype, but is preferably human IgG1.

Antibodies exist for example, as intact immunoglobulins or can be cleaved into a number of well-characterized fragments produced by digestion with various peptidases, such as papain or pepsin (see FIG. 2). Pepsin digests an antibody below the disulfide linkages in the hinge region to produce a F(ab)′₂ fragment of the antibody which is a dimer of the Fab composed of a light chain joined to a V_{H}-C_{H}1 by a disulfide bond. The F(ab)′₂ may be reduced under mild conditions to break the disulfide linkage in the hinge region thereby converting the F(ab)′₂ dimer to a Fab′ monomer. The Fab′ monomer is essentially an Fab with part of the hinge region (FIG. 2). See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

As used herein, an antibody can also be a single-chain antibody (scFv), which generally comprises a fusion polypeptide consisting of a variable domain of a light chain fused via a polypeptide linker to the variable domain of a heavy chain.

As used herein, “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation which is unique to the immune system of the host. Generally, an epitope consists of at least five such amino acids, and more usually consists of at least 8-10 such amino acids. Methods for determining the spatial conformation of such amino acids are known in the art.

5.1.1.1 Immunogen Production

An immunogen, typically the antigen to be cleared from a subject, is used to prepare antibodies by immunizing a suitable subject, (e.g., rabbit, goat, mouse or other mammal). An appropriate immunogenic preparation can contain, for example, recombinantly expressed or chemically synthesized antigen. The preparation can further include an adjuvant, such as Freund's complete or incomplete adjuvant, or similar immunostimulatory agent.

Isolated antigens to be used as immunogens, as well as isolated antigenic fragments, are suitable for use as immunogens to raise antibodies directed against an antigen. An isolated antigenic fragment suitable for use as an immunogen comprises at least a portion of the antigen that is 8 amino acids, more preferably 10 amino acids and more preferably still, 15 amino acids long.

In another embodiment, the antigen for use as an immunogen can be isolated from cells or tissue sources by an appropriate purification scheme using standard purification techniques. In another embodiment, immunogenic antigens are produced by recombinant DNA techniques. Alternative to recombinant expression, an antigen can be synthesized chemically using standard peptide synthesis techniques.

An “isolated” antigen is substantially free of cellular material or other contaminating material from the cell or tissue source from which the protein is derived, or substantially free of chemical precursors or other chemicals when chemically synthesized. The language “substantially free of cellular material” includes preparations of antigen in which the antigen is separated from cellular components of the cells from which it is isolated or recombinantly produced. Thus, an antigen that is substantially free of cellular material includes preparations of antigen having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the antigen. Accordingly such preparations of the antigen have less than about 30%, 20%, 10%, 5% (by dry weight) of chemical precursors or compounds other than the polypeptide of interest.

The invention also provides chimeric or fusion antigens for use as immunogens. As used herein, a “chimeric antigen” or “fusion antigen” comprises all or part of an antigen for use in the invention, operably linked to a heterologous polypeptide. Within the fusion antigen, the term “operably linked” is intended to indicate that the antigen and the heterologous polypeptide are fused in-frame to each other. The heterologous polypeptide can be fused to the N-terminus or C-terminus of the antigen.

One useful fusion antigen is a GST fusion antigen in which the antigen is fused to the C-terminus of GST sequences. Such fusion antigens can facilitate the purification of a recombinant antigens.

In another embodiment, the fusion antigen contains a heterologous signal sequence at its N-terminus so that the antigen can be secreted and purified to high homogeneity in order to produce high affinity antibodies. For example, the native signal sequence of an immunogen can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal and the protein A secretory signal (Pharmacia Biotech; Piscataway, N.J.).

In yet another embodiment, the fusion antigen is an immunoglobulin fusion protein in which all or part of an antigen is fused to sequences derived from a member of the immunoglobulin protein family. The immunoglobulin fusion proteins can be used as immunogens to produce antibodies directed against an antigen in a subject and to potentially purify additional antigens.

Chimeric and fusion proteins can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (e.g., Ausubel et al., supra). Moreover, many expression vectors are commercially available that already encode a fusion domain (e.g., a GST polypeptide). A nucleic acid encoding an immunogen can be cloned into such an expression vector such that the fusion domain is linked in-frame to the polypeptide.

5.1.1.2 Antibody Production

Antibodies can be prepared by immunizing a suitable subject with an antigen as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see generally, U.S. Pat. No. 5,914,112, which is incorporated herein by reference in its entirety.)

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay(ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal-antibody-secreting hybridomas, a monoclonal antibody directed against a pathogen or pathogenic antigenic molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of an immunogen.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif. (see, for example, U.S. Pat. No. 5,985,615)) and Medarex, Inc. (Princeton, N.J.), can be engaged to provide human antibodies directed against a selected antigen using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) antigen Bio/technology 12:899-903).

A pre-existing antibody directed against a pathogen can be used to isolate additional antigens of the pathogen by standard techniques, such was affinity chromatography or immunoprecipitation for use as immunogens. Moreover, such an antibody can be used to detect the protein (e.g., in a cellular lysate or cell supernatant) in order to evaluate the abundance and pattern of expression of the pathogen. The antibodies can also be used diagnostically to monitor pathogen levels in tissue as part of a clinical testing procedure, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Antibodies that are commercially available can be purchased and used to generate bispecific antibodies, e.g., from ATCC. In a preferred embodiment of the invention, the antibody is produced by a commercially available hybridoma cell line. In a more preferred embodiment, the hybridoma secretes a human antibody.

5.1.2 Bispecific Antibody Production and Purification

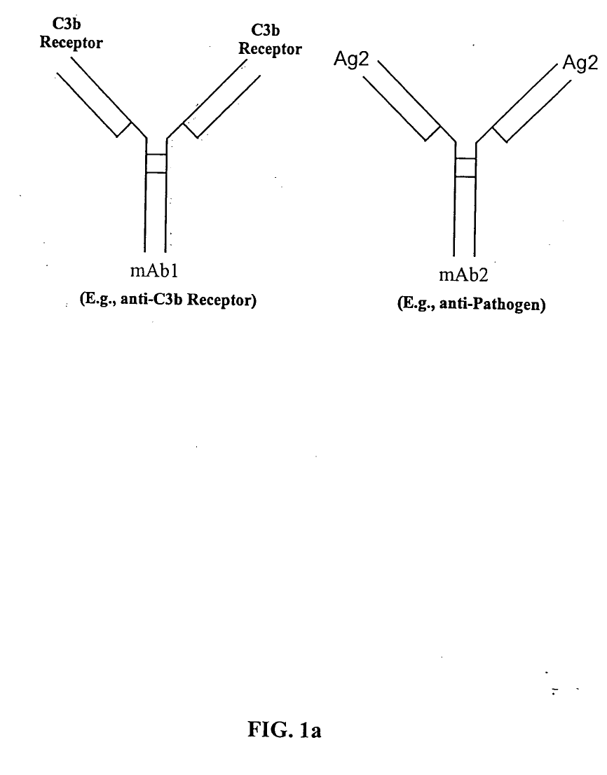

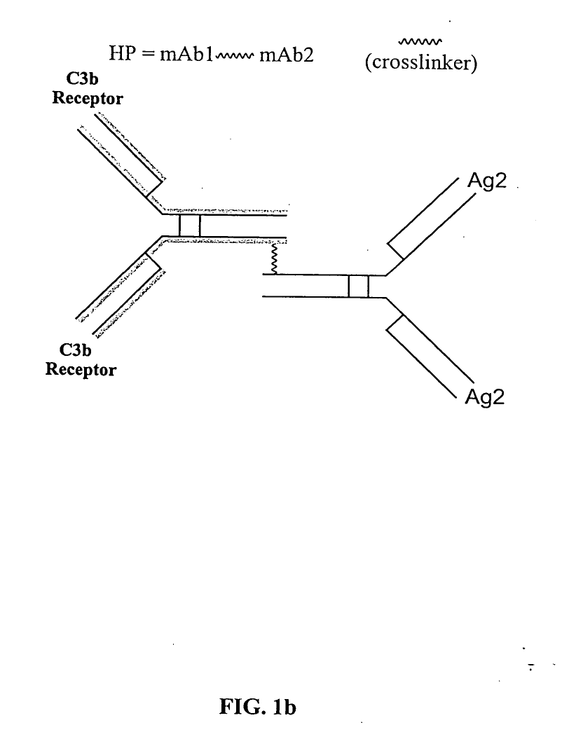

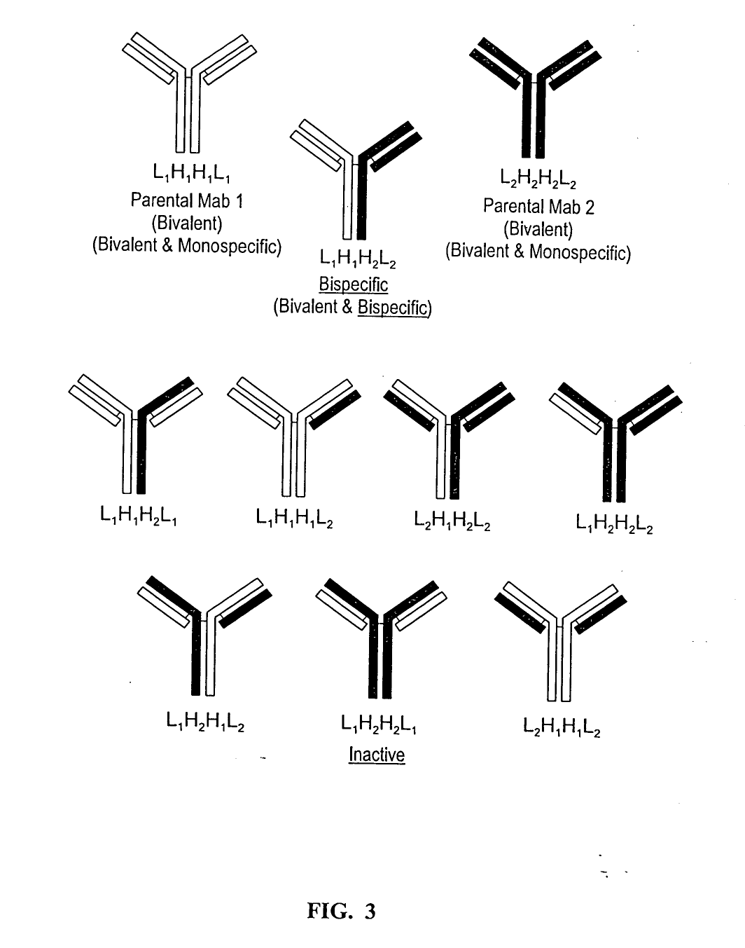

Production of full length bispecific antibodies is based on the coexpression of two immunoglobulin heavy chain-light chain pairs in a single hybridoma cell line, where two sets of antibody encoding genes encode for antibodies having different antigen specificities (Milstein et al., 1983, Nature, 305:537-539; FIG. 1, panel A). Because of the random assortment of immunoglobulin heavy and light chains, these hybridomas (i.e., ‘quadromas’) produce a potential mixture of 10 different antibody molecules (FIG. 3), of which only one has the correct bispecific structure (L₁H₁H₂L₂ of FIG. 3; FIG. 1, Panel C). Purification of the correct molecule, which is usually done by affinity chromatography steps, is rather cumbersome, and the product yields are low. Alternative purification procedures are disclosed in WO 93/08829, published 13 May 1993, and in Traunecker et al., 1991, EMBO J., 10:3655-3659.

The invention thus provides method of producing a bispecific immunoglobulin-secreting cell comprising the steps of: (a) fusing a first cell expressing an immunoglobulin which binds to a C3b-like receptor with a second cell expressing an immunoglobulin which binds to a pathogenic antigenic molecule; and (b) selecting for cells that express a bispecific immunoglobulin that comprises a first binding domain which binds to a C3b-like receptor, and a second binding domain which binds to a pathogenic antigenic molecule.

In a specific embodiment, a bispecific immunoglobulin of the invention is produced recombinantly (see, e.g., U.S. Pat. No. 4,816,397 dated Mar. 28, 1989 by Boss).

Thus, the invention provides a method for producing a bispecific molecule comprising a first binding domain which binds a C3b-like receptor and a second binding domain which binds a pathogenic antigenic molecule in a cell, comprising the steps of: (a) transforming a cell with one or more first DNA sequences encoding at least the first binding domain and one or more second DNA sequences encoding at least the second binding domain; and (b) expressing said first DNA sequences and said second DNA sequences so that said first and second binding domains are produced as separate molecules which assemble together in said transformed cell, whereby a bispecific molecule is formed that (i) does not consist of a first monoclonal antibody to CR1 that has been chemically cross-linked to a second monoclonal antibody, (ii) binds the C3b-like receptor, and (iii) binds the pathogenic antigenic molecule.

The invention also provides a method for producing a bispecific molecule comprising a first binding domain which binds a C3b-like receptor and a second binding domain which binds a pathogenic antigenic molecule in a cell, comprising the steps of: (a) transforming a first cell with one or more first DNA sequences encoding at least the first binding domain; (b) transforming a second cell with one or more second DNA sequences encoding at least the second binding domain; (c) expressing said first DNA sequences and said second DNA sequences so that said first and second binding domains are produced separately; (d) isolating said first and second binding domains; and (e) combining said first and second binding domains in vitro to form a bispecific molecule that binds the C3b-like receptor and binds the pathogenic antigenic molecule by contacting said first and second binding domains, and wherein the bispecific molecule does not consist of a first monoclonal antibody to CR1 that has been chemically cross-linked to a second monoclonal antibody. As used herein, “contacting” refers to the placing or mixing of two or more reactant molecules in a reaction buffer, e.g., in a liquid solution, such that the two or more reactant molecules can encounter and react.

The invention further provides a cell transformed with a first nucleotide sequence encoding a first binding domain and a second nucleotide sequence encoding a second binding domain, wherein when expressed in the cell, the two binding domains associate together to form a bispecific molecule, wherein the first binding domain binds a C3b-like receptor, and the second binding domain binds a pathogenic antigenic molecule, and wherein the bispecific molecule does not consist of a first monoclonal antibody to CR1 that has been chemically cross-linked to a second monoclonal antibody.

In one embodiment, the bispecific antibodies are produced recombinantly, whereby nucleotides which encode antibody variable domains with the desired binding specificities (antibody-antigen combining sites) are fused to nucleotides which encode immunoglobulin constant domain sequences. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and heavy chain (see, Arathoon et al., WO 98/50431).

DNAs encoding the immunoglobulin heavy chain fusions and, if desired, the immunoglobulin light chain, are inserted into separate expression vectors, and are co-transfected into a suitable host organism. This provides for the ability to adjust the proportions of each of the three polypeptide fragments in unequal ratios of the three polypeptide chains, thus providing optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm fused to the constant CH2 and CH3 domains, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3,1994.

The bispecific molecules comprising single polypeptides can be produced recombinantly using any standard method known in the art. In one embodiment, the nucleic acid encoding an antigen recognition region, e.g., an scFv, is fused to the nucleic acid encoding an antigen recognition region that binds a C3b-like receptor to obtain a fusion nucleic acids encoding a single polypeptide bispecific molecule. The nucleic acid is then expressed in a suitable host to produce the bispecific molecule.

For further details of generating bispecific antibodies see, for example, Suresh et al., 1986, Methods in Enzymology, 121:210. Using such techniques, a bispecific antibody which combines an anti-C3b-like receptor antibody (Nickells et al., 1998, Clin. Exp. Immunol. 112:27-33) and an antibody specific for an antigen can be prepared for use in the treatment of disease as defined herein (see, FIG. 1, panels A and C).

In another preferred embodiment, a bispecific antibody fraagent can be prepared by any one of the following non-limiting examples. For example, Fab′ fragments recovered from E. coli can be chemically coupled in vitro to form antibodies. See, Shalaby et al., 1992, J. Exp. Med., 175:217-225. Various techniques exist for making and isolating bispecific antibody fragments directly from recombinant cell culture. For example, heterodimers have been produced using leucine zippers (Kostelny et al., 1992, J. Immunol. 148:1547-1553). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers.

The “diabody” technology described by Hollinger et al., (1993, Proc. Natl. Acad. Sci. USA, 90:6444-6448) reported an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites (i.e., bispecific). In a similar protocol, Gruber et al. report the production of bispecific antibody fragments using only single-chain Fv (scFv) dimers (1994, J. Immunol., 152:5368).

### 5.2. Purification/Isolation of Bispecific Antibodies

In a preferred embodiment, bispecific antibodies secreted from the antibody secreting cells are isolated by ion exchange chromatography (See Section 6.2). Non-limiting examples of columns suitable for isolation of the bispecific antibodies of the invention include DEAE, Hydroxylapatite, Calcium Phosphate (Staerz and Bevan, 1986, Proc. Natl. Acad. Sci., 83:1453-1457).

In another preferred embodiment, properly fused cells (hybrid-hybridomas) are selected using fluorescent activated cell sorting (FACS). For example, before fusion, each hybridoma is grown in media with label, such as fluorescein isothiocyanate (FITC) or tetramethyl rhodamine isothiocyanate (TRITC). The first hybridoma is grown with a marker that is different from the second hybridoma. The cells are then fused by conventional methods and the bispecific antibody producing cells are identified and selected using FACS by measuring the fluorescent color of the cells (see Koolwijk et al., 1988, Hybridoma 7:217-225; or Karawajew et al., 1987, J. Immun. Methods, 96:265-270).

In another embodiment, bispecific antibodies secreted from the antibody secreting cells are isolated by three-step successive affinity chromatography (Corvalan and Smith, 1987, Cancer Immunol. Immunother., 24:127-132): the first column is made of protein A bound to a solid matrix, where the Fc portion of the antibody binds protein A, and wherein the antibodies bind the column; followed by a second column that utilizes C3b-like receptor binding to a solid matrix which assays for C3b-like receptor binding via a first variable domain; and followed by a third column that utilizes specific binding of an antigen of interest bound by a second variable domain.

In yet another embodiment, bispecific antibodies secreted from the antibody secreting cells are isolated by isoelectric focusing of antibodies. The skilled artisan will recognize that any method of purifying proteins using size or affinity will be suitable in the present invention.

5.2.1 Other Bispecific Molecules

Other bispecific molecules are within the scope of the invention and can be made using techniques well known in the art of molecular biology. In particular, cloning of DNAs can be performed as taught by Current Protocols in Molecular Biology, Ausubel et al., eds., John Wiley & Sons, 1992. Expression of recombinant proteins is also well known in the art.

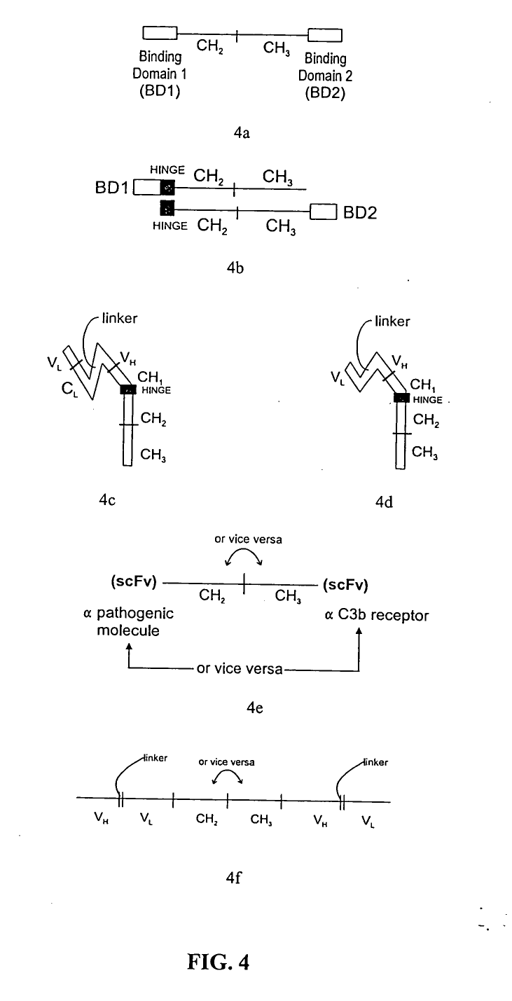

In one embodiment, the bispecific molecule of the invention is a single molecule (preferably a polypeptide) which consists essentially of, or alternatively comprises, a first binding domain (BD1) bound to the amino terminus of a CH2 and CH3 portion of an immunoglobulin heavy chain (Fc) bound to a second binding domain (BD2) at the Fc domain's carboxy terminus (FIG. 4, Panel A). In another embodiment, the CH2 domain and the CH3 domain positions are present in reverse order. One of the binding domains binds a C3b-like receptor, and the other of the binding domains binds a pathogenic antigenic molecule. The binding domains can individually be a scFv (i.e., a V_{L} fused via a polypeptide linker to a V_{H}) or a receptor or ligand or binding domain thereof, or other binding partner, with the desired specificity. For example, the binding domain that binds the pathogenic antigenic molecule can be a cellular receptor for a virus (e.g., CD4 and/or a chemokine receptor, which bind to HIV), or a receptor for a bacteria (e.g., polymyxin or multimers thereof which bind to Gram-negative bacteria), or a cellular receptor for a drug or other molecule (e.g., α domain of the IgE receptor which binds IgE, to treat or prevent allergic reactions), or a receptor for an autoantibody (e.g., acetylcholine receptor, for treating or preventing myasthenia gravis).

In an embodiment where a binding domain is not a polypeptide or is not otherwise readily expressed as a fusion protein with the other portions of the bispecific molecule, such binding domain can be cross-linked to the rest of the bispecific molecule. For example, polymyxin can be crosslinked to a fusion polypeptide comprising CH₂CH₃ and the binding domain that binds a C3b-like receptor.

In another embodiment, the bispecific molecule of the invention is a dimeric molecule consisting of a first molecule (preferably a polypeptide) consisting essentially of, or comprising, a BD1 bound to the amino terminus of an immunoglobulin Fc domain (a hinge region, a CH2 domain and a CH3 domain), and a second molecule (preferably a polypeptide), consisting essentially of, or comprising, a Fc domain with a BD2 domain bound to the Fc domain's carboxy terminus (FIG. 4, Panel B), wherein the Fc domains of the first and second polypeptides are complementary to and can associate with each other. BD1 and BD2 are as described above.

In a specific embodiment, one or both of the monomers of the bispecific molecule depicted in FIG. 4B has the structure depicted in FIG. 4C. FIG. 4C depicts a molecule (preferably a polypeptide) consisting essentially of, or comprising, a variable light chain domain (VL) and constant light chain domain (CL) followed by a linker molecule (of any structure/sequence) bound to the amino terminus of a variable heavy chain domain, followed by a CH1 domain, a hinge region, a CH2 domain, and a CH3 domain (FIG. 4, Panel C).

In another specific embodiment, one or both of the monomers depicted in FIG. 4B has the structure depicted in FIG. 4D. FIG. 4D depicts a molecule (preferably a polypeptide) consisting essentially of, or comprising, a scFv bound to the amino terminus of a CH1 domain, followed by a hinge region, a CH2 domain and a CH3 domain (FIG. 4, Panel D).

In another embodiment, the bispecific molecule of the invention is a molecule comprising two separate scFv with specificity for two separate antigens (one of which is the C3b-like receptor, the other of which is the pathogenic antigenic molecule). The molecule (preferably polypeptide) consists essentially of, or comprises, a first scFv domain bound to a CH2 domain, followed by a CH3 domain, and a second scFv domain (FIG. 4, Panel E).

In another embodiment, the bispecific molecule of the invention is a molecule consisting essentially of, or comprising, two variable regions with specificity for the two separate antigens. The molecule (preferably polypeptide) consists essentially of, or comprises, a first variable heavy chain domain bound to a variable light chain domain, followed by a CH2 domain, a CH3 domain, a variable heavy chain domain, and a variable light chain domain (FIG. 4, Panel F).

Furthermore, the invention also encompasses rearrangement of the position of any of the individual components of the bispecific molecules, wherein the bispecific molecule retains the ability to clear pathogenic antigenic molecules from the circulation. For example, the position of two binding domains (BD1 and BD2) may be switched for the bispecific molecule depicted in FIG. 4, Panels B, E and F. Alternatively, the positions of the CH2 and CH3 domains may be switched from that depicted in FIGS. 4A-4F. Further, the invention contemplates that the domains may be further rearranged into different positions relative to one another, while retaining its functional properties, i.e., binding to a C3b-like receptor, binding to a pathogenic antigenic molecule, and capable of being cleared from the circulation by macrophages. Moreover, as will be clear from the discussion above, the binding domains described above preferably, but need not be, polypeptides (including peptides). Moreover, the binding domains can provide the desired binding specificity via covalent or noncovalent linkage to the appropriate structure that mediates binding. For example, the binding domain may contain avidin or streptavidin that is noncovalently bound to a biotinylated molecule that in turn binds a pathogen antigenic molecule.

The foregoing bispecific molecules are preferably obtained by recombinant expression of genetically engineering nucleic acid constructs encoding the bispecific molecules, which can be made using methods well known in the art and/or described in Section 5.1.1 and its subsections above, and/or extracellular crosslinking methodology.

### 5.3 Polycolonal Populations of Bispecific Molecules

As used herein, a polyclonal population of bispecific molecules of the present invention refers to a population of bispecific molecules, said population comprising a plurality of different bispecific molecules each having a first antigen recognition region that binds a pathogenic antigenic molecule and a second antigen recognition region that binds a C3b-like receptor, wherein the first antigen recognition regions in the plurality of different bispecific molecules are each different and each have a different binding specificity and wherein each of said bispecific molecules does not consist of a first monoclonal antibody that has been chemically crosslinked to a second monoclonal antibody to CR1. In some embodiments, the first and second antigen recognition regions of each bispecific molecule in the polyclonal population do not comprise more than one heavy and light chain pair. Preferably, the plurality of bispecific molecules of the polyclonal population includes specificities for different epitopes of an antigenic molecule and/or for different variants of an antigenic molecule. More preferably, the plurality of bispecific molecules of the polyclonal population includes specificities for the majority of naturally-occurring epitopes of an antigenic molecule and/or for all variants of an antigenic molecule. The polyclonal population can also include specificities for a mixture of different antigenic molecules. In preferred embodiments, at least 90%, 75%, 50%, 20%, 10%, 5%, or 1% of bispecific molecules in the polyclonal population target the desired antigenic molecule and/or antigenic molecules. In other preferred embodiments, the proportion of any single bispecific molecule in the polyclonal population does not exceed 90%, 50%, or 10% of the population. The polyclonal population comprises at least 2 different bispecific molecules with different specificities. More preferably, the polyclonal population comprises at least 10 different bispecific molecules with different specificities. Most preferably, the polyclonal population comprises at least 100 different bispecific molecules with different specificities.

The polyclonal populations of bispecific molecules of the invention can be used for more efficient clearance of pathogens that have multiple epitopes and/or pathogens that have multiple variants or mutants, which normally cannot be effectively targeted and cleared by a monoclonal antibody having a single specificity. By targeting multiple epitopes and/or multiple variants of a pathogen, the polyclonal population of bispecific molecules is advantageous in the clearance of pathogens that have a higher mutation rate because simultaneous mutations at more than one epitopes tend to be much less frequent.

The polyclonal populations of bispecific molecules of the invention can comprise any type of bispecific molecules described previously in Sections 5.1, and 5.2. The polyclonal populations of bispecific molecules are produced by adapting any methods described in Sections 5.1, and 5.2.

The polyclonal population of bispecific molecules of the present invention can be produced by transfecting a hybridoma cell line that expresses an immunoglobulin that binds a C3b-like receptor with a population of eukaryotic expression vectors containing nucleic acids encoding the heavy and light chain variable regions of a polyclonal population of immunoglobulins that bind different antigenic molecules. Cells that express bispecific immunoglobulins that comprise a first binding domain which binds to a pathogenic antigenic molecule and a second binding domain which binds to a C3b-like receptor are then selected using standard methods known in the art. The polyclonal population of immunoglobulins can be obtained by any method known in the art, e.g., from a phage display library. If a phage display library is used, the number of specificities of such phage display library is preferably near the number of different specificities that are expressed at any one time by lymphocytes. More preferably the number of specificities of the phage display library is higher than the number of different specificities that are expressed at any one time by lymphocytes. Most preferably the phage display library comprises the complete set of specificities that can be expressed by lymphocytes. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In a preferred embodiment, the polyclonal population of eukaryotic expression vectors is produced from a phage display library according to Den et al., 1999, J. Immunol. Meth. 222:45-57. The phage display library is screened to select a polyclonal sublibrary having binding specificities directed to the antigenic molecule or antigenic molecules of interests by affinity chromatography (McCafferty et al., 1990, Nature 248:552; Breitling et al., 1991, Gene 104:147; and Hawkins et al., 1992, J. Mol. Biol. 226:889). The nucleic acids encoding the heavy and light chain variable regions are then linked head to head to generate a library of bidirectional phage display vectors. The bidirectional phage display vectors are then transferred in mass to bidirectional mammalian expression vectors (Sarantopoulos et al., 1994, J. Immunol. 152:5344) which are used to transfect the hybridoma cell line.

In other preferred embodiments, the polyclonal population of bispecific molecules is produced by a method using the whole collection of selected displayed antibodies without clonal isolation of individual members as described in U.S. Pat. No. 6,057,098, which is incorporated by reference herein in its entirety. Polyclonal antibodies are obtained by affinity screening of a phage display library having a sufficiently large repertoire of specificities with an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. In one embodiment, the population of expression vectors is then co-expressed with vectors containing a nucleotide sequence encoding an anti-CR1 binding domain in a suitable host. In another embodiment, the population of expression vectors and the vectors containing a nucleotide sequence encoding an anti-CR1 binding domain are expressed in separate hosts and the antigen binding domains and the anti-CR1 binding domain are combined in vitro to form the polyclonal population of bispecific molecules.

In still other embodiments, the polyclonal populations of bispecific antibodies are produced recombinantly, whereby the polyclonal population of nucleic acids which encode antibody variable domains with the desired binding specificities (antibody-antigen combining sites) are fused to nucleotides which encode immunoglobulin constant domain sequences. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and heavy chain (see, Arathoon et al., WO 98/50431).

DNAs encoding the immunoglobulin heavy chain fusions and, if desired, the immunoglobulin light chain, are inserted into separate expression vectors, and are co-transfected into a suitable host organism. This provides for the ability to adjust the proportions of each of the three polypeptide fragments in unequal ratios of the three polypeptide chains, thus providing optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, each bispecific molecule in the polyclonal population is composed of a hybrid immunoglobulin heavy chain with a different first binding specificity in one arm fused to the constant CH2 and CH3 domains, and a hybrid immunoglobulin heavy chain-light chain pair (providing a second binding specificity) in the other arm. It was found that this asymmetric structure facilitates the separation of the desired bispecific compounds from unwanted immunoglobulin chain combinations, as the presence of an immunoglobulin light chain in only one half of the bispecific molecule provides for a facile way of separation. This approach is disclosed in WO 94/04690 published Mar. 3, 1994.

Polyclonal populations of bispecific molecules comprising single polypeptide bispecific molecules can be produced recombinantly. A polyclonal population of nucleic acids encoding a polyclonal population of selected antigen recognition regions is fused to nucleic acids encoding the antigen recognition region that binds a C3b-like receptor to obtain a population of fusion nucleic acids encoding a population of bispecific molecules. The population of nucleic acids are then expressed in a suitable host to produce a polyclonal population of bispecific molecules. In a preferred embodiment, the polyclonal population of nucleic acids encoding a polyclonal library of selected antigen recognition regions are obtained according to the method described in U.S. Pat. No. 6,057,098.

In still other preferred embodiments, the polyclonal population of bispecific molecules is produced from a population of displayed antibodies obtained by affinity screening with a set of antigens, such as but are not limited to a set of variants of a pathogen and/or a mixture of various pathogens. Such polyclonal population of bispecific molecules can be used to target and clear a set of antigens.

The polyclonal populations of bispecific molecules can be purified using any methods known in the art. The content of a polyclonal population of bispecific molecules can be determined using standard methods known in the art.

Although polyclonal populations of bispecific molecules produced from phage display libraries are described, it will be recognized by one skilled in the art that the plurality of second antigen recognition portions used in the generation of a population can be obtained from any population of suitable antigen recognition moieties. Populations of bispecific molecules produced from such population of antigen recognition moieties are intended to be within the scope of the invention.

### 5.4. Cocktails of Bispecific Molecules

Various purified bispecific molecules can be combined into a “cocktail” of bispecific molecules. As used herein, a cocktail of bispecific molecules of the present invention refers to a mixture of purified bispecific molecules for targeting one or a mixture of antigens. In particular, the cocktail of bispecific molecules refers to a mixture of purified bispecific molecules having a plurality of first antigen binding domains that target different or same antigenic molecules and that are of mixed types. For example, the mixture of the first antigen binding domains can be a mixture of peptides, nucleic acids, and/or organic small molecules. A cocktail of bispecific molecules is generally prepared by mixing various purified bispecific molecules. Such bispecific molecule cocktails are useful, inter alia, as personalized medicine tailored according to the need of individual patients.

### 5.5. Target Pathogenic Antigenic Molecules

The present invention provides methods of treating or preventing a disease or disorder associated with the presence of a pathogenic antigenic molecule. The pathogenic antigenic molecule can be any substance that is present in the circulation that is potentially injurious to or undesirable in the subject to be treated, including but not limited to proteins or drugs or toxins, autoantibodies or autoantigens, or a molecule of any infectious agent or its products. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

Circulating pathogenic antigenic molecules cleared by the fixed tissue phagocytes include any antigenic moiety that is harmful to the subject. Examples of harmful pathogenic antigenic molecules include any pathogenic antigen associated with a parasite, fungus, protozoa, bacteria, or virus. Furthermore, circulating pathogenic antigenic molecules may also include toxins, immune complexes, autoantibodies, drugs, an overdose of a substance, such as a barbiturate, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The present invention further provides an embodiment for treating transplantation rejection comprising administering to a subject an effective amount of a bispecific antibody that will bind and remove immune cells or factors involved in transplantation rejection, e.g., transplantation antigen specific antibodies.

5.5.1 Autoimmune Antigens

In one embodiment, the pathogenic antigenic molecule to be cleared from the circulation includes autoimmune antigens. These antigens include but are not limited to autoantibodies or naturally occurring molecules associated with autoimmune diseases.

Many different autoantibodies can be cleared from the circulation of a primate by using the bispecific antibodies of the present invention. In a non-limiting example, IgE (immunoglobulin E) antibodies are cleared from the circulation by the bispecific antibodies of the invention. More specifically, the bispecific antibodies comprise one variable region that is specific to an IgE and a second variable region that is specific to a C3b-like receptor. This bispecific antibody can be used to decrease circulating IgE antibodies thereby reducing or inhibiting allergic reactions such as asthma.

In another example, certain humans with hemophilia have been shown to be deficient in factor VIII. Recombinant factor VIII replacement treats this hemophilia. However, eventually some patients develop antibodies against factor VIII, thus interfering with the therapy. The bispecific antibodies of the present invention prepared with an anti-anti-factor VIII antibodies provides a therapeutic solution for this problem. In particular, a bispecific antibody with specificity of the first variable region to anti-factor VIII autoantibodies and specificity of the second variable region to C3b-like receptor would be therapeutically useful in clearing the autoantibodies from the circulation, thus, ameliorating the disease.

Further examples of autoantibodies which can be cleared by the bispecific antibodies of the present invention include, but are not limited to, autoantibodies to the following antigens: the muscle acetylcholine receptor (the antibodies are associated with the disease myasthenia gravis); cardiolipin (associated with the disease lupus); platelet associated proteins (associated with the disease idiopathic thrombocytopenic purpurea); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is characterized and is associated with disease pathogenesis.

When the above bispecific antibodies are injected into the circulation of a human or non-human primate, the bispecific antibodies will bind to red blood cells via the human or primate C3b receptor variable domain recognition site, at a high percentage and in agreement with the number of C3b-like receptor sites on red blood cells. The bispecific antibodies will simultaneously associate with the autoantibody indirectly, through the antigen, which is bound to the monoclonal antibody. The red blood cells which have the bispecific antibody/autoantibody complex on their surface then facilitate the neutralization and clearance from the circulation of the bound pathogenic autoantibody.

In the present invention, the bispecific antibodies facilitate pathogenic antigen or autoantibody binding to hematopoietic cells expressing a C3b-like receptor on their surface and subsequently clear the pathogenic antigen or autoantibody from the circulation, without also clearing the hematopoietic cells.

5.5.2 Infectious Diseases

In specific embodiments, infectious diseases are treated or prevented by administration of a bispecific molecule that binds both an antigen of an infectious disease agent and a C3b-like receptor. Thus, in such an embodiment, the pathogenic antigenic molecule is an antigen of an infectious disease agent.

Such antigen can be but is not limited to: influenza virus hemagglutinin (Genbank accession no. JO2132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24 M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g. , infectious laryngotracheitis virus glycoprotein G or glycoprotein I), a glycoprotein of La Crosse virus (Gonzales-Scarano et al., 1982, Virology 120: 42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, NY, p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323 A published Jun. 4, 1980; Ganem and Varmus, 1987, Ann. Rev. Biochem. 56:651-693; Tiollais et al., 1985, Nature 317:489-495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Additional diseases or disorders that can be treated or prevented by the use of a bispecific molecule of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses

Bacterial diseases or disorders that can be treated or prevented by the use of bispecific molecules of the present invention include, but are not limited to, Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, and Bacillus anthracis (anthrax), etc.

Protozoal diseases or disorders that can be treated or prevented by the use of bispecific molecules of the present invention include, but are not limited to, plasmodia, eimeria, Leishmania, and trypanosoma.

5.5.3 Additional Pathogenic Antigenic Molecules

In one embodiment, the pathogenic antigenic molecule to be cleared from the circulation by the methods and compositions of the present invention encompass any serum drug, including but not limited to barbiturates, tricyclic antidepressants, and Digitalis.

In another embodiment, the pathogenic antigenic molecule to be cleared includes any serum antigen that is present as an overdose and can result in temporary or permanent impairment or harm to the subject. This embodiment particularly relates to drug overdoses.

In another embodiment, the pathogenic antigenic molecule to be cleared from the circulation include naturally occurring substances. Examples of naturally occurring pathogenic antigenic molecules that could be removed by the methods and compositions of the present invention include but are not limited to low density lipoproteins, interleukins or other immune modulating chemicals and hormones.

### 5.6. Dose of Bispecific Antibodies

The dose can be determined by a physician upon conducting routine experiments. Prior to administration to humans, the efficacy is preferably shown in animal models. Any animal model for a circulatory disease known in the art can be used.

More particularly, the dose of the bispecific antibody can be determined based on the hematopoietic cell concentration and the number of C3b-like receptor epitope sites bound by the anti-C3b-like receptor monoclonal antibodies per hematopoietic cell. If the bispecific antibody is added in excess, a fraction of the bispecific antibody will not bind to hematopoietic cells, and will inhibit the binding of pathogenic antigens to the hematopoietic cell. The reason is that when the free bispecific antibody is in solution, it will compete for available pathogenic antigen with bispecific antibody bound to hematopoietic cells. Thus, the bispecific antibody-mediated binding of the pathogenic antigens to hematopoietic cells follows a bell-shaped curve when binding is examined as a function of the concentration of the input bispecific antibody concentration.

Viremia may result in up to 10⁸-10⁹ viral particles/ml of blood (HIV is 10⁶/ml; (Ho, 1997, J. Clin. Invest. 99:2565-2567)); the dose of therapeutic bispecific antibodies should preferably be, at a minimum, approximately 10 times the antigen number in the blood.

In general, for antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of bispecific antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a bispecific antibody can include a single treatment or, preferably, can include a series of treatments. In a preferred example, a subject is treated with a bispecific antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a bispecific antibody, used for treatment may increase or decrease over the course of a particular treatment. Changes in dosage may result and become apparent from the results of diagnostic assays as described herein.

It is understood that appropriate doses of bispecific antibody agents depends upon a number of factors within the ken of the ordinarily skilled physician, veterinarian, or researcher. The dose(s) of the bispecific antibody will vary, for example, depending upon the identity, size, and condition of the subject or sample being treated, further depending upon the route by which the composition is to be administered, if applicable, and the effect which the practitioner desires the bispecific antibody to have upon a pathogenic antigenic molecule or autoantibody.

It is also understood that appropriate doses of bispecific antibodies depend upon the potency of the bispecific antibody with respect to the antigen to be cleared. Such appropriate doses may be determined using the assays described herein. When one or more of these bispecific antibodies is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the biopecific antibody employed, the age, body weight, general health, gender, and diet of the subject, the time of administration, the route of administration, the rate of excretion, any drug combination, and the concentration of antigen to be cleared.

### 5.7. Pharmaceutical Formulation and Administration

The bispecific antibodies of the invention can be incorporated into pharmaceutical compositions suitable for administration. Such compositions typically comprise bispecific antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the bispecific antibody, use thereof in the compositions is contemplated. Supplementary bispecific antibodies can also be incorporated into the compositions.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that the viscosity is low and the bispecific antibody is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the bispecific antibody (e.g., one or more bispecific antibodies) in the required amount in an appropriate solvent with one or a combination of ingredients enumerated above, as required, followed by filtered sterilization. Generally, dispersions are prepared by incorporating the bispecific antibody into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the bispecific antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of bispecific antibody calculated to produce the desired therapeutic effect in association with the required pharmaceutical carrier. The specification for the dosage unit forms of the invention are dictated by and directly dependent on the unique characteristics of the bispecific antibody and the particular therapeutic effect to be achieved, and the limitations inherent in the art of compounding such a bispecific antibody for the treatment of individuals.

The pharmaceutical compositions can be included in a kit, in a container, pack, or dispenser together with instructions for administration.

### 5.8. Kits

The invention also provides kits containing the bispecific molecules of the invention, or one or more nucleic acids encoding polypeptide bispecific molecules of the invention, or cells transformed with such nucleic acids, in one or more containers. The nucleic acids can be integrated into the chromosome, or exist as vectors (e.g., plasmids, particularly plasmid expression vectors). Kits containing the pharmaceutical compositions of the invention are also provided.

### 5.9. Ex Vivo Preparation of the Bispecific Molecule

In an alternative embodiment, the bispecific molecule, such as a bispecific antibody, is prebound to hematopoietic cells of the subject ex vivo, prior to administration. For example, hematopoietic cells are collected from the individual to be treated (or alternatively hematopoietic cells from a non-autologous donor of the compatible blood type are collected) and incubated with an appropriate dose of the therapeutic bispecific antibody for a sufficient time so as to allow the antibody to bind the C3b-like receptor on the surface of the hematopoietic cells. The hematopoietic cell/bispecific antibody mixture is then administered to the subject to be treated in an appropriate dose (see, for example, Taylor et al., U.S. Pat. No. 5,487,890).

The hematopoietic cells are preferably blood cells, most preferably red blood cells.

Accordingly, in a specific embodiment, the invention provides a method of treating a mammal having an undesirable condition associated with the presence of a pathogenic antigenic molecule, comprising the step of administering a hematopoietic cell/bispecific molecule complex to the subject in a therapeutically effective amount, said complex consisting essentially of a hematopoietic cell expressing a C3b-like receptor bound to one or more bispecific molecules, wherein said bispecific molecule (a) does not consist of a first monoclonal antibody to CR1 that has been chemically cross-linked to a second monoclonal antibody, (b) comprises a first binding domain which binds the C3b-like receptor on the hematopoietic cell, and (c) comprises a second binding domain which binds the pathogenic antigenic molecule. The method alternatively comprises a method of treating a mammal having an undesirable condition associated with the presence of a pathogenic antigenic molecule comprising the steps of (a) contacting a bispecific molecule with hematopoietic cells expressing a C3b-like receptor, to form a hematopoietic cell/bispecific molecule complex, wherein the bispecific molecule (i) does not consist of a first monoclonal antibody to CR1 that has been chemically cross-linked to a second monoclonal antibody, (ii) comprises a first binding domain which binds the C3b-like receptor, and (iii) comprises a second binding domain which binds the pathogenic antigenic molecule; and (b) administering the hematopoietic cell/bispecific molecule complex to the mammal in a therapeutically effective amount.

The invention also provides a method of making a hematopoietic cell/bispecific molecule complex comprising contacting a bispecific molecule with hematopoietic cells that express a C3b-like receptor under conditions conducive to binding, such that a complex forms, said complex consisting essentially of a hematopoietic cell bound to one or more bispecific molecules, wherein said bispecific molecule (a) comprises a first binding domain that binds the C3b-like receptor on the hematopoietic cells, (b) comprises a second binding domain that binds a pathogenic antigenic molecule, and (c) does not consist of a first monoclonal antibody to CR1 that has been chemically cross-linked to a second monoclonal antibody.

Taylor et al. (U.S. Pat. No. 5,879,679, hereinafter “the '679 patent”) have demonstrated in some instances that the system saturates because the concentration of autoantibodies (or other pathogenic antigen) in the plasma is so high that even at the optimum input of bispecific antibodies, not all of the atitoantibodies can be bound to the hematopoietic cells under standard conditions. For example, for a very high titer of autoantibody sera, a fraction of the autoantibody is not bound to the hematopoietic cells due to its high concentration.

However, saturation can be solved by using combinations of bispecific antibodies which contain monoclonal antibodies that bind to different sites on a C3b-like receptor. For example, the monoclonal antibodies 7G9 and 1B4 bind to separate and non-competing sites on the primate C3b receptor. Therefore, a “cocktail” containing a mixture of two bispecific antibodies, each made with a different monoclonal antibody to the C3b-like receptor, may give rise to greater binding of antibodies to red blood cells. The bispecific antibodies of the present invention can also be used in combination with certain fluids used for intravenous infusions.

In yet another embodiment, the bispecific molecule, such as a bispecific antibody, is prebound to red blood cells in vitro as described above, using a “cocktail” of at least two different bispecific antibodies. In this embodiment, the two different bispecific antibodies bind to the same antigen, but also bind to distinct and non-overlapping recognition sites on the C3b-like receptor. By using at least two non-overlapping bispecific antibodies for binding to the C3b-like receptor, the number of bispecific antibody-antigen complexes that can bind to a single red blood cell is increased. Thus, by allowing more than one bispecific antibody to bind to a single C3b-like receptor, antigen clearance is enhanced, particularly in cases where the antigen is in very high concentrations (see for example the '679 patent, column 6, lines 41-64).

### 6. EXAMPLE

The following example describes the production of a specific hybrid hybridoma resulting in the production of a bispecific antibody. One of ordinary skill in the relevant art will recognize that any hybridoma that secretes an antibody with specificity to an antigen can be used in the present invention. Additionally, the following example utilizes an antibody purification scheme involving hydroxylapatite chromatography and isoelectric focusing, however, one of ordinary skill in the relevant art will recognize that any purification scheme according to the invention would be suitable.

Approximately 25% of the U.S. population suffers from an atopic disease. Genetic and environmental factors induce individuals to synthesize allergen-specific IgE that attaches to circulating basophils and tissue mast cells through a high affinity receptor. Binding of the receptor by IgE induces release of preformed agents such as histamine and other allergic reaction mediators. The ensuing allergic reaction can lead to chronic inflammation of the airways resulting in, among other symptoms, rhinitis and asthma. Therefore, the control of IgE concentration, or removal of IgE provides a potential method to alleviate allergic diseases (Saini et al., 1999, J. Immunology, 162:5624-5630).

### 6.1. Fusion of Two Hybridomas

Two hybridomas are fused together in order to obtain a hybrid hybridoma that secretes an antibody with specificity to both a primate C3b receptor and also to IgE. The hybridoma 7G9 secretes a mouse monoclonal antibody with specificity to the human C3b receptor (see the '679 patent). The hybridoma MAE11 secretes a mouse monoclonal antibody with specificity to IgE (Jardieu and Fick, 1999, Allergy and Immun., 118:112-115). The two hybridoma cell lines are grown in conventional media prior to fusion.

Fusion is performed after the two 7G9 and MAE11 hybridomas are grown to log phase in Dulbecco's Modified Eagle's Medium (DMEM). For fusion, equal numbers of cells in 50 ml of DMEM, i.e., 5×10⁷ cells, are mixed with 1 ml of 45% polyethylene glycol and 10% dimethyl sulfoxide. After a fixed period of time, the cells are centrifuged at low speed and resuspended in DMEM absent fusion reagents. An aliquot is cloned on the same day on soft agarose at four dilutions. About 100 clones are expanded on 24 well plates with 10% DMEM. Supernatants are assayed for antibody production and the best producers are recloned and expanded using normal tissue culture procedures.

The assay for antibody production requires spotting on a 1×1 cm sheet of nitrocellulose (hereinafter “the square”) approximately 100 micrograms of the antigen, in the first case, the C3b receptor. The square is dried for about five minutes and blocked with 5% BSA in PBS for at least ten minutes. About 2 to 5 microliters of the hybridoma secretion is spotted on the square. After 2 to 5 minutes, the square is washed with PBS and incubated with a 1 to 5000 dilution of 2 to 5 microliters of goat-anti-mouse antibodies conjugated to horse radish peroxidase. After 2 to 5 minutes the square is washed with PBS three times for at least 5 minutes per wash and developed with 0.4 mg of 4-chloro-1-naphthol per ml/0.03% H₂O₂.

A color reaction indicates binding to the antigen and indicates the cloned hybridoma is positive for secretion of an anti-C3b receptor antibody. Positive clones are then tested for expression of anti-IgE antibodies using the same protocol where IgE is the test antigen. Hybridomas simultaneously positive for both antigens are expanded in liquid culture and stocks are frozen.

### 6.2. Pufification of Bispecific Antibodies

The following protocol describes a method to purify bispecific antibodies from ascites but can also be used with tissue culture supernatants. The bispecific antibodies are purified from secreted non-specific antibodies and secreted proteins using ion exchange chromatography (Suresh and Milstein, 1986, Methods in Enzymology, 121:210).

Analysis of ascites or concentrated culture supernatants by cellulose acetate electrophoresis in 0.04 M veronal buffer (pH 8.6) using a Beckman microzone electrophoresis apparatus typically exhibits three prominent immunoglobulin bands. The middle band is the bispecific antibody and the other two bands represent the parental antibodies.

First, ascites is collected and clarified by centrifugation to remove cells and other particulate matter. The ascites is diluted 1:1 with saline. An equal volume of saturated ammonium sulfate is added gradually, over one hour, with stirring to achieve a 50% salt saturation. The precipitate is dissolved in a minimum amount of PBS and exhaustively dialyzed with two changes in 100 volumes of 10 mM sodium phosphate buffer at pH 7.5.

Next, the dialyzed crude antibody is fractionated on a DEAE column to obtain relatively pure bispecific antibodies. A DE-52 (Whatman, microgranular form) column is prepared measuring approximately 2×9 cm for processing of 8 to 10 ml of ascites or 2 liters of serum free supernatant. The column is equilibrated by washing in 50 bed volumes of 10 mM sodium phosphate pH 7.5. The crude antibody is loaded and fractions collected. A UV monitor continuously records the effluent absorption and the column is washed with 1 bed volume of 10 mM sodium phosphate pH 7.5.

Finally, the antibody is eluted by connecting the column to a linear gradient of 10 to 100 mM sodium phosphate pH 7.5. Ideally, three peaks are obtained and the middle peak is the bispecific antibody. The purity of the fractions are analyzed by SDS-PAGE and silver staining. Antigen binding activity is tested as described in Section 6.1 above.

The present invention is not to be limited in scope by the specific embodiments described herein. Indeed, various modifications of the invention in addition to those described herein will become apparent to those skilled in the art from the foregoing description and accompanying drawings. Such modifications are intended to fall within the scope of the appended claims.

Various references are cited herein above, including patent applications, patents, and publications, the disclosures of which are hereby incorporated by reference in their entireties for all purposes.

What is claimed is:

1. A bispecific molecule that
(a) comprises a first binding domain which binds a pathogenic antigenic molecule;
(b) comprises a second binding domain which binds a C3b-like receptor; and
(c) does not consist of a first monoclonal antibody to CR1 that has been chemically cross-linked to a second monoclonal antibody.

2. The bispecific molecule of claim 1 that is a bispecific immunoglobulin, wherein the first binding domain is a first immunoglobulin variable region, and the second binding domain is a second immunoglobulin variable region.

3. The bispecific molecule of claim 1 that is a molecule which consists essentially of
(a) said first or said second binding domain, bound to
(b) a polypeptide consisting of (i) a CH₂ domain followed by a CH₃ domain, or (ii) a CH₃ domain followed by a CH₂ domain, bound to
(c) said second binding domain when (a) is said first binding domain, or said first binding domain when (a) is said second binding domain.

4. The bispecific molecule of claim 1 that is a dimeric molecule consisting of (a) a first molecule consisting essentially of a said first or second binding domain bound to the amino terminus of a first immunoglobulin Fc domain; and (b) a second molecule consisting essentially of a second immunoglobulin Fc domain bound at its carboxy-terminus to (i) said second binding domain when said first binding domain is present in said first molecule, or (ii) said first binding domain when said second binding domain is resent in said first molecule; wherein the first and second Fc domains are complementary to and associate with each other.

5. The bispecific molecule of claim 1 that is a dimeric molecule comprising two polypeptides, each independently selected from the group consisting of (a) a first polypeptide consisting essentially of, in amino- to carboxy-terminal order, an immunoglobulin variable light chain domain, an immunoglobulin constant light chain domain, a linker polypeptide, an immunoglobulin variable heavy chain domain, a CH1 domain, an immunoglobulin hinge region, a CH2 domain, and a CH3 domain; and (b) a second polypeptide consisting essentially of, in amino- to carboxy-terminal order, a scFv, a CH1 domain, an immunoglobulin hinge region, a CH2 domain, and a CH3 domain.

6. The bispecific molecule of claim 1 that is a polypeptide that consists essentially of, in amino- to carboxy-terminal order, a first scFv, a CH2 domain, a CH3 domain, and a second scFv domain.

7. The bispecific molecule of claim 1 that is a polypeptide that consists essentially of, in amino- to carboxy-terminal order, a first scFv, a CH3 domain, a CH2 domain, and a second scFv domain.

8. The bispecific molecule of claim 1 that is a polypeptide that consists essentially of, in amino- to carboxy-terminal order, a first immunoglobulin variable heavy chain, a first immunoglobulin variable light chain, a CH2 domain, a CH3 domain, a second immunoglobulin variable heavy chain, and a second immunoglobulin variable light chain.

9. The bispecific molecule of claim 1 or 2 that is purified.

10. The bispecific molecule of any of claims 1-8 wherein the pathogenic antigenic molecule is an antigen of an infectious agent.

11. The bispecific molecule of any of claims 1-8 wherein the pathogenic antigenic molecule is an autoantibody.

12. The bispecific molecule of claim 1 that is a polypeptide.

13. The bispecific molecule of claim 3 or 4 that is a polypeptide.

14. A nucleic acid encoding the bispecific molecule of claim 12.

15. A nucleic acid encoding the bispecific molecule of any of claims 2, and 5-10.

16. A cell transformed with the nucleic acid of claim 14.

17. The nucleic acid of claim 14 that is isolated.

18. The nucleic acid of claim 14 that is present in a plasmid expression vector.

19. A kit comprising in one or more containers, one or more isolated nucleic acids encoding the bispecific molecule of claim 2.

20. A kit comprising in one or more contained a cell transformed with one or more nucleic acids encoding the ispecific molecule of claim 2.

21. A method of treating a mammal having an undesirable ondition associated with the presence of a pathogenic antigenic molecule comprising administering to the mammal a therapeutically effective dose of a bispecific molecule, which bispecific molecule (a) does not consist of a first monoclonal antibody to CR1 that has been chemically cross-linked to a second monoclonal antibody, (b) comprises a first binding domain which binds said pathogenic antigenic molecule, and (c) comprises a second binding domain which binds a C3b-like receptor of the mammal.

22. The method of claim 21 wherein the bispecific molecule is a bispecific immunoglobulin, that has a first variable region that binds the pathogenic antigenic molecule and a second variable region that binds the C3b-like receptor.

23. The method of claim 21 wherein the bispecific molecule is a fragment of a bispecific immunoglobulin that has a first variable region that binds the pathogenic antigenic molecule and a second variable region that binds a C3b-like receptor expressed on a cell.

24. The method of claim 21, 22 or 23 wherein the bispecific molecule is 90% cleared from the circulation of the mammal within 48 hours.

25. The method of claim 21, 22 or 23, wherein said administering is intravenous.

26. The method of claim 21, 22 or 23, wherein said mammal is a human, and said C3b-like receptor is CR1.

27. The method of claim 21, 22 or 23, wherein said mammal is a non-human mammal.

28. The method of claim 21, 22 or 23, wherein the pathogenic antigenic molecule is a protein of a pathogen.

29. The method of claim 21, 22 or 23, wherein the pathogenic antigenic molecule is an autoantibody of an autoimmune disorder.

30. The method of claim 21, 22 or 23, wherein the pathogenic antigenic molecule is an antigen of an infectious agent that causes the undesirable condition.

31. The method of claim 21, 22 or 23, wherein the pathogenic antigenic molecule is a drug that causes the undesirable condition.

32. The method of claim 30 wherein the infectious agent is a virus.

33. The method of claim 30 wherein the infectious agent is a bacterium.

34. The method of claim 30 wherein the infectious agent is a fungus.

35. The method of claim 30 wherein the infectious agent is a protozoan.

36. The method of claim 30 wherein the infectious agent is a parasite.

37. A pharmaceutical composition comprising a purified bispecific molecule of claim 1, 2 or 3, in an amount effective to treat a mammal having an undesirable condition associated with the presence of the pathogenic antigenic molecule, and a pharmaceutically acceptable carrier.

38. The pharmaceutical composition of claim 37 wherein the pathogenic antigenic molecule is an infectious agent of a mammal.

39. A kit comprising in a container a bispecific molecule that (a) does not consist of a first monoclonal antibody to CR1 that has been chemically cross-linked to a second monoclonal antibody, (b) comprises a first binding domain which binds a pathogenic antigenic molecule, and (c) comprises a second binding domain which binds a C3b-like receptor.

40. The kit of claim 39 wherein the pathogenic antigenic molecule is an antigen of an infectious agent.

41. The kit of claim 39 wherein the infectious agent is a virus.

42. The kit of claim 39 wherein the infectious agent is a bacterium.

43. The kit of claim 39 wherein the infectious agent is a fungus.

44. The kit of claim 39 wherein the infectious agent is a protozoan.

45. The kit of claim 39 wherein the infectious agent is a parasite.

46. The kit of claim 39 wherein the pathogenic antigenic molecule is a drug.

47. The kit of claim 39 wherein the pathogenic antigenic molecule is an autoimmune antigen.

48. The kit of claim 39 wherein the pathogenic ntigenic molecule is a low density lipoprotein.

49. A method for producing a bispecific molecule omprising a first binding domain which binds a C3b-like receptor and a second binding domain which binds a pathogenic antigenic molecule in a cell, comprising the steps of:
(a) transforming a cell with a one or more first DNA sequences encoding at least the first binding domain and a one or more second DNA sequences encoding at least the second binding domain; and
(b) expressing said first DNA sequences and said second DNA sequences so that said first and second binding domains are produced as separate molecules which assemble together in said transformed cell, whereby a bispecific molecule is formed that (i) does not consist of a first monoclonal antibody to CR1 that has been chemically cross-linked to a second monoclonal antibody, (ii) binds the C3b-like receptor, and (iii) binds the pathogenic antigenic molecule.

50. A method for producing a bispecific molecule comprising a first binding domain which binds a C3b-like receptor and a second binding domain which binds a pathogenic antigenic molecule in a cell, comprising the steps of:
(a) transforming a first cell with one or more first DNA sequences encoding at least the first binding domain;
(b) transforming a second cell with one or more second DNA sequences encoding at least the second binding domain;
(c) expressing said first DNA sequences and said second DNA sequences so that said first and second binding domains are produced separately;
(d) isolating said first and second binding domains; and
(e) combining said first and second binding domains in vitro to form a bispecific molecule that binds the C3b-like receptor and binds the pathogenic antigenic molecule, and wherein the bispecific molecule does not consist of a first monoclonal antibody to CR1 that has been chemically cross-linked to a second monoclonal antibody.

51. The method of claim 49, wherein the bispecific molecule is a bispecific immunoglobulin or fragment thereof that comprises (a) a first binding domain formed by a first immunoglobulin variable light chain domain and a first immunoglobulin variable heavy chain domain, which binds the C3b-like receptor, and (b) a second binding domain formed by a second immunoglobulin variable light chain domain, and a second immunoglobulin variable heavy chain domain, which binds the pathogenic antigenic molecule.

52. The method of claim 51, wherein the first DNA sequences and the second DNA sequences are present in different vectors.

53. The method of claim 49, 50 or 51, wherein the first DNA sequences and the second DNA sequences are present in a single vector.

54. The method of claim 52, wherein each vector is a plasmid expression vector.

55. The method of claim 51, wherein the first and second variable light chain domains and first and second variable heavy chain domains of the first and second binding domains are all on separate immunoglobulin chains that are expressed and assembled together in the cell and secreted therefrom as an immunologically functional molecule.

56. The method of claim 50, wherein the first binding domain is produced in insoluble or membrane bound form and is solubilized and allowed to refold in solution to form an immunologically functional antigen binding molecule or fragment thereof.

57. The method of claim 51, wherein said first or said second DNA sequences further encode at least one constant domain, wherein the constant domain is derived from a source different from that from which the variable domain to which it is attached is derived.

58. The method of claim 51, wherein said first and second DNA sequences are derived from one or more monoclonal antibody producing hybridomas.

59. A cell transformed with a first nucleotide sequence encoding a first binding domain and a second nucleotide sequence encoding a second binding domain, wherein when expressed in the cell, the two binding domains associate together to form a bispecific molecule, wherein the first binding domain binds a C3b-like receptor, and the second binding domain binds a pathogenic antigenic molecule, and wherein the bispecific molecule does not consist of a first monoclonal antibody to CR1 that has been chemically crosslinked to a second monoclonal antibody.

60. A method of producing a bispecific immunoglobulin-secreting cell comprising the steps of:
(a) fusing a first cell expressing an immunoglobulin which binds to a C3b-like receptor with a second cell expressing an immunoglobulin which binds to a pathogenic antigenic molecule; and
(b) selecting for cells that express a bispecific immunoglobulin that comprises a first binding domain which binds to a C3b-like receptor, and a second binding domain which binds to a pathogenic antigenic molecule.

61. A nucleic acid encoding the bispecific molecule of claim 13.

62. A cell transformed with the nucleic acid of claim 61.

63. A method of preventing an undesirable condition associated with the presence of a pathogenic antigenic molecule in a mammal, comprising administering prior to the onset of the undesirable condition, to the mammal a prophylactically effective amount of a bispecific molecule, which bispecific molecule (a) does not consist of a first monoclonal antibody to CR1 that has been chemically cross-linked to a second monoclonal antibody, (b) comprises a first binding domain which binds said pathogenic antigenic molecule, and (c) comprises a second binding domain which binds a C3b-like receptor of the mammal.

64. The method of claim 63 wherein the bispecific molecule is a bispecific monoclonal antibody.

65. A bispecific antibody producing cell produced by the method of claim 61.

66. The bispecific antibody producing cell of claim 65, wherein the cell is a mouse cell.

67. The bispecific antibody producing cell of claim 65, wherein the cell is a human cell.

68. A method of treating a mammal having an undesirable condition associated with the presence of a pathogenic antigenic molecule comprising the steps of:
(a) contacting a bispecific molecule with hematopoietic cells expressing a C3b-like receptor, to form a hematopoietic cell/bispecific molecule complex, wherein the bispecific molecule (i) does not consist of a first monoclonal antibody to CR1 that has been chemically cross-linked to a second monoclonal antibody, (ii) comprises a first binding domain which binds the C3b-like receptor, and (iii) comprises a second binding domain which binds the pathogenic antigenic molecule; and
(b) administering the hematopoietic cell/bispecific molecule complex to the mammal in a therapeutically effective amount.

69. A method of treating a mammal having an undesirable condition associated with the presence of a pathogenic antigenic molecule, comprising the step of administering a hematopoietic cell/bispecific molecule complex to the subject in a therapeutically effective amount, said complex consisting essentially of a hematopoietic cell expressing a C3b-like receptor bound to one or more bispecific molecules, wherein said bispecific molecule (a) does not consist of a first monoclonal antibody to CR1 that has been chemically cross-linked to a second monoclonal antibody, (b) comprises a first binding domain which binds the C3b-like receptor on the hematopoietic cell, and (c) comprises a second binding domain which binds the pathogenic antigenic molecule.

70. A cell that secretes the bispecific molecule of claim 1 or 2.

71. A kit comprising in one or more containers a first vector and a second vector, said first vector comprising a first DNA sequence encoding at least a first immunoglobulin variable heavy chain domain fused via a polypeptide linker to a first immunoglobulin variable light chain domain, and said second vector comprising a second DNA sequence encoding at least a second immunoglobulin variable heavy chain domain fused via a polypeptide linker to a second immunoglobulin ariable light chain domain, wherein said first immunoglobulin variable heavy chain domain and said first mmunoglobulin variable light chain bind a pathogenic ntigenic molecule, and said second immunoglobulin variable heavy chain domain and second immunoglobulin variable light hain domain bind a C3b-like receptor.

72. A method of making a hematopoietic cell/bispecific molecule complex comprising contacting a bispecific molecule with hematopoietic cells that express a C3b-like receptor under conditions conducive to binding, such that a complex forms, said complex consisting essentially of a hematopoietic cell bound to one or more bispecific molecules, wherein said bispecific molecule (a) comprises a first binding domain that binds the C3b-like receptor on the hematopoietic cells, (b) comprises a second binding domain that binds a pathogenic antigenic molecule, and (c) does not consist of a first monoclonal antibody to CR1 that has been chemically cross-linked to a second monoclonal antibody.

73. The method of claim 21 wherein the bispecific molecule is a molecule which consists essentially of
(a) said first or said second binding domain, bound to
(b) a polypeptide consisting of (i) a CH₂ domain followed by a CH₃ domain, or (ii) a CH₃ domain followed by a CH₂ domain, bound to
(c) said second binding domain when (a) is said first binding domain, or said first binding domain when (a) is said second binding domain.

74. The method of claim 21 wherein the bispecific molecule is a dimeric molecule consisting of (a) a first molecule consisting essentially of a said first or second binding domain bound to the amino terminus of a first immunoglobulin Fc domain; and (b) a second molecule consisting essentially of a second immunoglobulin Fc domain bound at its carboxy-terminus to (i) said second binding domain when said first binding domain is present in said first molecule, or (ii) said first binding domain when said second binding domain is present in said first molecule; wherein the first and second Fc domains are complementary to and associate with each other.

75. The method of claim 21 wherein said first and second binding domains are each a single chain Fv.

76. The method of claim 49, wherein said first DNA sequences or said second DNA sequences further encode at least one constant domain, wherein the constant domain is derived from a source different from that from which the variable domain to which it is attached is derived.

77. The method of claim 49, wherein said first DNA sequences and said second DNA sequences are derived from different monoclonal antibody producing hybridomas.

78. A bispecific immunoglobulin which comprises a first binding domain which binds to a C3b-like receptor and a second binding domain which binds to a pathogenic antigenic molecule, produced by the method comprising the steps of:
(a) fusing a first cell expressing an immunoglobulin which binds to a C3b-like receptor with a second cell expressing an immunoglobulin which binds to a pathogenic antigenic molecule;
(b) selecting for cells that express a bispecific immunoglobulin that (i) binds to the C3b-like receptor and (ii) binds to the pathogenic antigenic molecule;
(c) culturing cells selected in step (b); and
(d) recovering the bispecific immunoglobulin expressed by the cultured cells.

79. A hematopoietic cell/bispecific molecule that consists essentially of a hematopoietic cell bound to one or more bispecific molecules, wherein each of said bispecific molecules (a) comprises a first binding domain which binds a C3b-like receptor on the hematopoietic cell, (b) comprises a second binding domain which binds a pathogenic antigenic molecule, and (c) does not consist of a first monoclonal antibody to CR1 that has been chemically cross-linked to a second monoclonal antibody.

80. A method of producing a bispecific molecule comprising culturing the cell of claim 16 under conditions such that the encoded bispecific molecule is expressed by the cell, and recovering the expressed bispecific molecule.

81. A polyclonal population of bispecific molecules comprising a plurality of bispecific molecules each comprising (a) a different first antigen recognition region, and (b) a second antigen recognition region that binds a C3b-like receptor, said different first antigen recognition regions having different binding specificities, wherein each of said bispecific molecules in said plurality does not consist of a first monoclonal antibody that has been chemically cross-linked to a second monoclonal antibody to CR1.

82. A composition comprising a plurality of purified bispecific molecules, wherein each bispecific molecule of said plurality of purified bispecific molecules comprises a first antigen recognition region that binds a C3b-like receptor and a second antigen recognition region that binds a pathogenic antigenic molecule, said plurality of purified bispecific molecules each comprising a different second antigen recognition portions that has a different binding specificity, wherein each of said bispecific molecules in said plurality does not consist of a first monoclonal antibody that has been chemically cross-linked to a second monoclonal antibody to CR1.

83. The polyclonal population of bispecific molecules of claim 81, wherein each bispecific molecule in said plurality consists essentially of
(a) said first or said second antigen recognition region, bound to
(b) a polypeptide consisting of (i) a CH₂ domain followed by a CH₃ domain, or (ii) a CH₃ domain followed by a CH₂ domain, bound to
(c) said second antigen recognition region when (a) is said first antigen recognition region, or said first antigen recognition region when (a) is said second antigen recognition region.

84. The polyclonal population of bispecific molecules of claim 81, wherein each bispecific molecule in said plurality is a dimeric molecule consisting of (a) a first molecule consisting essentially of a said first or second binding domain bound to the amino terminus of a first immunoglobulin Fc domain; and (b) a second molecule consisting essentially of a second immunoglobulin Fc domain bound at its carboxy-terminus to (i) said second binding domain when said first binding domain is present in said first molecule, or (ii) said first binding domain when said second binding domain is present in said first molecule; wherein the first and second Fc domains are complementary to and associate with each other.

85. The polyclonal population of bispecific molecules of claim 81, wherein each bispecific molecule in said plurality is a dimeric molecule comprising two polypeptides, each independently selected from the group consisting of (a) a first polypeptide consisting essentially of, in amino- to carboxy-terminal order, an immunoglobulin variable light chain domain, an immunoglobulin constant light chain domain, a linker polypeptide, an immunoglobulin variable heavy chain domain, a CH1 domain, an immunoglobulin hinge region, a CH2 domain, and a CH3 domain; and (b) a second polypeptide consisting essentially of, in amino- to carboxy-terminal order, a scFv, a CH1 domain, an immunoglobulin hinge region, a CH2 domain, and a CH3 domain.

86. The polyclonal population of bispecific molecules of claim 81, wherein each bispecific molecule in said plurality is a polypeptide that consists essentially of, in amino- to carboxy-terminal order, a first scFv, a CH2 domain, a CH3 domain, and a second scFv domain.

87. The polyclonal population of bispecific molecules of claim 81, wherein each bispecific molecule in said plurality is a polypeptide that consists essentially of, in amino- to carboxy-terminal order, a first scFv, a CH3 domain, a CH2 domain, and a second scFv domain.

88. The polyclonal population of bispecific molecules of claim 81, wherein each bispecific molecule in said plurality is a polypeptide that consists essentially of, in amino- to carboxy-terminal order, a first immunoglobulin variable heavy chain, a first immunoglobulin variable light chain, a CH2 domain, a CH3 domain, a second immunoglobulin variable heavy chain, and a second immunoglobulin variable light chain.

89. The polyclonal population of bispecific molecules of claim 81, wherein the pathogenic antigenic molecule is an antigen of an infectious agent.

90. The polyclonal population of bispecific molecules of claim 81, wherein the pathogenic antigenic molecule is an autoantibody.

91. The polyclonal population of bispecific molecules of claim 81, wherein each bispecific molecule in said plurality is a polypeptide.

92. A population of nucleic acids encoding the polyclonal population of bispecific molecules of claim 91.

93. A population of cells transformed with the nucleic acids of claim 92.

94. The population of nucleic acids of claim 92 that is a purified population.

95. The population of nucleic acids of claim 92 that is present in a population of eukaryotic expression vectors.

96. A kit comprising in one or more containers, the population of nucleic acids of claim 92.

97. A kit comprising in one or more contained a population of cells transformed with the population of nucleic acids of claim 92.

98. A method of treating a mammal having an undesirable condition associated with the presence of a pathogenic antigenic molecule comprising administering to the mammal a therapeutically effective dose of a polyclonal population of bispecific molecules comprising a plurality of bispecific molecules, each bispecific molecule in said plurality comprising (a) a different first antigen recognition region, and (b) a second antigen recognition region that binds a C3b-like receptor, said different first antigen recognition regions having different binding specificities, wherein each of said bispecific molecules in said plurality does not consist of a first monoclonal antibody that has been chemically cross-linked to a second monoclonal antibody to CR1.

99. The method of claim 98, wherein said administering is intravenous.

100. The method of claim 98, wherein said mammal is a human, and said C3b-like receptor is CR1.

101. The method of claim 98, wherein said mammal is a non-human mammal.

102. The method of claim 98, wherein the pathogenic antigenic molecule is a protein of a pathogen.

103. The method of claim 98, wherein the pathogenic antigenic molecule is an autoantibody of an autoimmune disorder.

104. The method of claim 98, wherein the pathogenic antigenic molecule is an antigen of an infectious agent that causes the undesirable condition.

105. The method of claim 98, wherein the pathogenic antigenic molecule is a drug that causes the undesirable condition.

106. The method of claim 104 wherein the infectious agent is a virus.

107. The method of claim 104 wherein the infectious agent is a bacterium.

108. The method of claim 104 wherein the infectious agent is a fungus.

109. The method of claim 104 wherein the infectious agent is a protozoan.

110. The method of claim 104 wherein the infectious agent is a parasite.

111. A pharmaceutical composition comprising a polyclonal population of bispecific molecules of claim 81, in an amount effective to treat a mammal having an undesirable condition associated with the presence of the pathogenic antigenic molecule, and a pharmaceutically acceptable carrier.

112. The composition of claim 82, wherein said plurality is present in an amount effective to treat a mammal having an undesirable condition associated with the presence of the pathogenic antigenic molecule, said composition further comprising a pharmaceutically acceptable carrier.

113. The composition of claim 111 wherein the pathogenic antigenic molecule is an infectious agent of a mammal.

114. The composition of claim 112 wherein the pathogenic antigenic molecule is an infectious agent of a mammal.

115. A method of producing a population of bispecific molecules, comprising transfecting a hybridoma cell line that expresses an immunoglobulin that binds a C3b-like receptor with a population of eukaryotic expression vectors containing nucleotide sequences encoding the heavy and light chain variable regions of a population of immunoglobulins that bind different antigenic molecules, and subjecting the transfected hybridoma cell line to conditions under which the nucleotide sequences are expressed such that a population of bispecific molecules is produced by the transfected hybridoma cell line, each bispecific molecule of said population having a first antigen recognition region that binds a pathogenic antigenic molecule and a second antigen recognition region that binds a C3b-like receptor.

116. The method of claim 115, wherein pairs of said nucleotide sequences encoding the heavy and light chain variable regions, respectively, are linked head to head to form bidirectional vectors.

117. A method of producing a population of bispecific molecules, comprising:
(a) selecting from a phage display library a plurality of phage that display antigen recognition polypeptides, each having a different respective binding specificity using affinity screening;
(b) obtaining a plurality of nucleic acids encoding said plurality of antigen recognition polypeptides, respectively;
(c) fusing each nucleic acid of said plurality of nucleic acids to nucleic acids which encode immunoglobulin constant domain sequences to produce a plurality of fusion nucleic acids encoding a plurality of fusion proteins each comprising an antigen recognition polypeptide fused to an immunoglobulin constant domain; and
(d) co-expressing said plurality of fusion nucleic acids in a host, to produce said polyclonal population of bispecific molecules;
wherein each member of said population has a first antigen recognition region that binds a pathogenic antigenic molecule and a second antigen recognition region that binds a C3b-like receptor.

118. A method of producing a polyclonal population of bispecific molecules, comprising:
(a) selecting from a phage display library a plurality of phage that display antigen recognition polypeptides, each having a different respective binding specificity using affinity screening;
(b) obtaining a plurality of nucleic acids encoding said plurality of antigen recognition polypeptides, respectively;
(c) fusing each nucleic acid of said plurality of nucleic acids to nucleic acids which encode immunoglobulin constant domain sequences to produce a plurality of fusion nucleic acids encoding a plurality of fusion proteins each comprising an antigen recognition polypeptide fused to an immunoglobulin constant domain;
(d) expressing said plurality of fusion nucleic acids in a first group of host cells to produce said plurality of fusion proteins;
(e) expressing nucleic acids encoding an antigen recognition region that binds a C3b-like receptor in a second group of host cells to produce said antigen recognition region; and
(f) contacting said produced fusion proteins and said produced antigen recognition region that binds a C3b-like receptor, to produce said polyclonal population of bispecific molecules;
each member of said polyclonal population having a first antigen recognition region that binds a pathogenic antigenic molecule and a second antigen recognition region that binds a C3b-like receptor.

119. A method of producing a polyclonal population of bispecific molecules, comprising:
(a) selecting from a phage display library a plurality of phage that display antigen recognition polypeptides, each having a different respective binding specificity using affinity screening;
(b) obtaining a plurality of nucleic acids encoding said plurality of antigen recognition polypeptides, respectively;
(c) fusing each nucleic acid of said plurality of nucleic acids to nucleic acids encoding the antigen recognition region that binds a C3b-like receptor to produce a plurality of fusion nucleic acids encoding a plurality of fusion proteins each comprising an antigen recognition polypeptide fused to an antigen recognition region that binds a C3b-like receptor; and
(d) expressing said plurality of fusion nucleic acids in a host, to produce said polyclonal population of bispecific molecules;
each member of said polyclonal population being a single chain polypeptide and having a first antigen recognition region that binds a pathogenic antigenic molecule and a second antigen recognition region that binds a C3b-like receptor.
